SEQUENCE LISTING (1 sequence)

SEQ ID NO: 1 - protein, 17 aa, Artificial Sequence
  Synthetic peptide
DENPVVHFFKNIVTPRT

(19) United States
(12) Patent Application Publication
(10) Pub. No.: US 2012/0022228 A1
(43) Pub. Date: Jan. 26, 2012

(54) PURIFICATION OF AMPHOTERIC PRODUCTS, OR OF PRODUCTS LIABLE TO BE CONVERTED INTO AMPHOTERIC PRODUCTS

(75) Inventors: Matthieu GIRAUD, Sion (CH); John MCGARRITY, Brig-Glis (CH); Jean-Hugues RENAULT, Cauroy Les Hermonville (FR); Leslie BOUDESOCQUE, Brimont (FR)

(73) Assignees: CENTRE NATIONAL DE LA RECHERCHE SCIENTIFIQUE, Paris Cedex 16 (FR); LONZA AG, Basel (CH); UNIVERSITE DE REIMS CHAMPAGNE-ARDENNE, Reims (FR)

(21) Appl. No.: 13/161,673

(22) Filed: Jun. 16, 2011

Related U.S. Application Data
(60) Provisional application No. 61/368,414, filed on Jul. 28, 2010.

(30) Foreign Application Priority Data
Jun. 18, 2010 (EP) 10 305 656.0

(51) Int. Cl. C07K 1/20

(52) U.S. Cl. 530/326; 530/417; 530/416; 562/433; 562/554

(57) ABSTRACT

A process for purifying at least one product from a substrate containing at least one product, includes subjecting the substrate to centrifugal partition chromatography in an ion exchange displacement mode to purify the at least one product from the substrate, the at least one product being an amphoteric product.

The invention relates to the purification of amphoteric products, or products liable to be converted into amphoteric products, by centrifugal partition chromatography.

Centrifugal Partition Chromatography (CPC) is a type of liquid-liquid hydrostatic chromatographic technique. The CPC column is constituted by a succession of chambers, or partition cells, linked to one-another, and engraved along a ring. A centrifugal force field is applied to the ring by triggering the rotation of the axis perpendicular to the ring plane.

CPC is a particular kind of liquid-liquid countercurrent chromatography in which a biphasic solvent system is used to partition analytes between two liquid phases in thermodynamic equilibrium. The CPC column (filled by the two equilibrated liquid phases of a biphasic system) is made of partition disks engraved to form partition cells that are connected together by capillary ducts. Thanks to a centrifugal force field, one liquid phase is maintained stationary inside the column while the other one is pumped through it to mobilize the analytes according to their partition behaviour (Berthod A., Countercurrent Chromatography—The Support-Free Liquid Stationary Phase, Comprehensive Analytical Chemistry, Elsevier Science B.V., Amsterdam 2002).

The CPC is different from the Counter Current Chromatography (CCC). The CCC columns are constituted by a helicoidal coil rotating around an axis in a planetary movement. The rotation axis and the axis of the coil are parallel. CCC is further described in Ito Y., Principle and instrumentation of countercurrent chromatography in Countercurrent chromatography—Theory and practice, Eds Mandava B, Ito Y., Marcel Dekker, Inc., New York, 1988, 3, 79-443

Displacement modes are development techniques used in purification by chromatography. The Displacement modes involve physical or chemical factors that favour the solubilisation of the products to be purified in one of the two phases (mobile or stationary), depending on this factor. Displacement mode increases the migrations of products from one phase to the other. It slows down the migration speed of the products along the column, and thus increases the resolution of the purification. The physical or chemical factor enabling the displacement mode is selected in order to interact with the product to be purified. In an appropriate enablement, some impurities are not affected by the displacement mode and thus migrate through the column faster than the product to be purified, allowing an improved purification.

In liquid-liquid support-free techniques, different displacement modes are known.

The pH-zone refining can be cited as an example of displacement mode using a physical factor, namely pH. pH refining zone displacement mode is not subject matter of the present invention, and is further described in Renault, J. H.; Nuzillard, J. M.; Le Crouérour, G.; Thëpenier, P.; Zèches-Hanrot, M.; Le Men-Olivier, L. J. Chromatogr. A 1999, 849, 421-431 and references therein; Y. Ito, K. Shinomiya, H. M. Fales, A. Weisz, A. L. Scher, in: W. D. Conway, 265 R. J. Petroski (Eds.), Modern Countercurrent Chromatography, American 266 Chemical Society, Washington, D.C., 1995, p. 156 (Ch. 14); A. Toribio, E. Delannay, B. Richard, K. Plé, M. Zèches-Hanrot, J.-M. Nuzillard, J.-H. Renault, Preparative isolation of huperzines A and B from Huperzia serrata by displacement centrifugal partition chromatography, J. Chromatogr. A, 2007, 1140, 101-106.

pH-zone refining is restricted to solutes showing a dramatic difference in polarity and, therefore, in solubility between their neutral and ionized forms. These limitations exclude the application of pH-zone refining to ionized, strictly water-soluble or amphoteric molecules.

The ion exchange can be cited as an example of displacement mode using a chemical factor. Ion exchange displacement mode relies on the ionic force of ions introduced in the column during the purification, or in other words, on the free enthalpy of formation (or affinity) of ion pairs involving the product to be purified, an ion pair being composed by a cation and an anion.

The purification of natural products using CPC in displacement mode has already been reported in the art. For example, one may refer to the purification of the cyclopeptide alkaloid Lotusine G (Le Crouéour et al., Fitoterapia, 2002, 73, 63-68) using CPC and pH-zone refining displacement mode, or to the purification of the plant metabolite sinalbin (Toribio et al., J. Sep. Sci., 2009, 32, 1801-1807) using CPC and ion-exchange displacement mode with an anionic exchanger.

Purification of peptides is industrially achieved by preparative chromatography (HPLC form example) using mainly reversed phase or ion exchange chromatographic support. All these techniques involve a solid chromatographic support both for the adsorption or the partition mode.

CPC is not used to purify peptides on an industrial scale, because of its loading capacity inferior to the above mentioned commonly used purification techniques.

Nevertheless, the Craig Apparatus which are only based on the partition of the analytes between two phases, are used to industrial production of peptides (http://www.theliquidphase.org/index.php?title=CCD)

Purification of complex peptide mixture is difficult to achieve. Peptides are fragile compounds that may denature or degrade under harsh chemical condition. Furthermore, the peptide amino-acid sequences may be closely related, thus providing compounds with almost similar physico-chemical properties which are very difficult to discriminate with common chromatographic techniques (Sewald N., Jakubke H. D., peptides: chemistry and biology, Wiley CH, Weinheim, 2005, 12-18).

One of the aims of the invention is to provide a method for the purification of amphoteric products or of products liable to be converted into amphoteric products (amphoteric convertible products).

One of the aims of the invention is to provide a method for the separation of peptide mixtures.

Another aim of the invention is to provide a method for the extraction of peptides from natural product extracts.

Another aim of the invention is to provide a method for the extraction of peptides from crude reaction mixtures.

The invention relates to the use of centrifugal partition chromatography in an ion exchange displacement mode for the implementation of a purification process of at least one product to be purified from a substrate containing it, said product to be purified being an amphoteric product or an amphoteric convertible product.

The invention relates to the use of centrifugal partition chromatography in an ion exchange displacement mode for the implementation of a purification process of at least one product to be purified from a substrate containing it, said product to be purified being an amphoteric product.

In the present invention, the Inventors have identified that the centrifugal partition chromatography associated with an ion exchange displacement mode is efficient in the purification of at least one product to be purified from a substrate containing it, said product to be purified being an amphoteric product or an amphoteric convertible product.

The term “purification from a substrate” designates the separation of at least one product from the other products contained in a substrate. The separation may be total or partial.

When the purification is total, the recovered product is pure.
When the purification is partial, one or several impurities are removed from the substrate but the product is obtained in a combination of at least two products.

The term “substrate” designates a mixture of chemical products from which at least one product is to be purified. The substrate may be a reaction mixture, this reaction mixture may be crude, filtrated, or even partially purified, a natural extract (from plants, bacteria, animal, or any type of cells); this natural extract may be crude, filtrated, or even partially purified.

The term “amphoteric” designates a product that can react either as an acid or as a base. Amphoteric products can either accept or donate a proton and thus can act as base or acid respectively.

The term “amphoteric convertible” designates a product that can become amphoteric after chemical transformation such as protecting group removal.

According to another embodiment, the invention relates to the use of centrifugal partition chromatography in an ion exchange displacement mode such as here above defined, wherein said substrate is contained in a chromatographic mixture, said chromatographic mixture containing also a solvent mixture, at least one displacer, at least one exchanger, and at least one retainer.

The term “chromatographic mixture” designates the liquid mixture present in the chromatography column when the purification process is triggered, and which contains the substrate. This chromatographic mixture comprises a solvent mixture, a mixture of displacer, of retainer and of exchanger.

The term “solvent mixture” designates a combination of solvents which form the purification media of the chromatography.

CPC is a liquid-liquid purification technique, thus all the components of the chromatographic mixture have to be either miscible or soluble in at least one of the solvents present in the column.

The displacer, retainer and exchanger are three ionic species that enable the displacement mode. The retainer and the displacer form respectively an ion pair with the exchanger.

The substrate, as defined above, designates a mixture of chemical products from which at least one product is to be purified. The substrate is soluble in the solvent mixture.

The exchanger also forms an ion pair with the product to be purified.

Thus, three distinct types of ion pairs are formed:
the pair between the at least one product to be purified and the exchanger,
the pair between the retainer and the exchanger, and
the pair between the displacer and the exchanger.

When an ion pair is formed, this formation releases a certain amount of energy that corresponds to the free enthalpy of formation of the ion pair. Each of the two constituents of this pair breaks the pair when it can form a new pair with another ion and when said new pair has a free enthalpy of formation superior to the enthalpy of formation of the previously formed pair

The ions pair free enthalpy of formation is an absolute value which is characterised by the two ions forming the ion pair.

Among all the ion pairs involving the exchanger, the ion pair with the highest enthalpy of formation is the “displacer exchanger” ion pair. This ion pair has the strongest affinity among the ion pairs involving the exchanger.

Among all the ion pairs involving the exchanger, the ion pair with the lowest enthalpy of formation is the “retainer exchanger” ion pair. This ion pair has the weakest affinity among the ion pairs involving the exchanger.

At the beginning of the purification, when no product to be purified has been injected in the column, the exchanger forms an ion pair with a retainer. This “exchanger—retainer” ion pair breaks up when placed in contact with the product to be purified, because the free enthalpy of formation of the “product to be purified exchanger” is superior to the free enthalpy of formation of the “exchanger—retainer” ion pair. In other words, the affinity of the product to be purified for the exchanger is higher, or superior, than the affinity of the retainer for the exchanger.

The driving force of the ion exchange displacement mode is the displacer. The displacer forms a displacer—exchanger ion pair with the exchanger, and said displacer—exchanger ion pair has a free enthalpy of formation superior to the one of any other ion pair present in the column. The displacer excludes all the other products forming ion pair with the exchanger, in the zone in which the displacer is present. Providing the displacer is continuously injected into the column during the purification process, the content of the portion of the column containing the displacer increases progressively.

A key feature of the ion exchange displacement mode is the cascade of ion pairs that are formed within the column. The ion pairs with a high affinity (or high free enthalpy of formation) are formed in priority over the ion pair with a low affinity (or low free enthalpy of formation), thus products to be purified that form ion pairs having a low free enthalpy of formation are salted-out of the stationary phase, and forced to migrate further in the column in order to form ion pair in a zone containing no product that may form an ion pair having a higher free enthalpy of formation.

Thus, products to be purified form ion pairs, which mutually exclude each other. The exclusion order is based on the affinity of the ion pair formed. A succession of ion pairs, all comprising the exchanger, is formed in the stationary phase, and this succession is called an “isotachtic train”. Each ion pair part of the isotachtic train constitutes a member of the isotachtic train.

The concepts of high or low affinities (free enthalpy of formation), are relative to the ionic species present in the chromatographic mixture. High or low affinity of an ion pair is defined with respect to another ion pair. In any case the limit values of affinity are given by the “retainer exchanger” ion pair (low affinity), and the “displacer exchanger” ion pair (high affinity).

FIG. 1 represents the formation of the isotachtic train when the substrate containing the products to be purified is introduced into the column.

FIG. 2 represents the isotachtic train when the displacer is introduced into the column

The isotachtic train is constituted by the three types of ion pairs, namely: the retainer—exchanger ion pair, the products to be purified—exchanger ion pairs, and the displacer—exchanger ion pair. This isotachtic train is formed in the stationary phase.

The products to be purified are introduced in the CPC column in the form of ion pairs with a counter ion. The displacer is introduced in the CPC column in the form of an ion pair with a counter ion. These counter ions may be the same or different chemical species.

Counter ions cannot form ion pairs with the exchanger because they have the same electrical charge.

Counter ions are soluble in the mobile phase. Counter ions can form ion pairs with respectively the retainer (“retainer—counter ion” ion pair), the products to be purified (“product to be purified—counter ion” ion pair), and the displacer (“displacer—counter ion” ion pair), when the retainer, products to be purified, and displacer are salted out of the stationary phase into the mobile phase and require an ion of opposite charge to keep the global electrostatic balance of the phase.

During the purification step, the displacer is constantly injected into the column. Progressively, as time passes and the amount of displacer injected in the column increases, all the other products present in the column are recovered at the output of the column. The products emerge at the output of the column, each product at a different time, the products emerging in the order of the isotachtic train that was formed in the column during the purification process.

According to another advantageous embodiment, the invention relates to the use of centrifugal partition chromatography in an ion exchange displacement mode such as here above defined wherein said product to be purified is at least one protein or at least one peptide or peptide derivative, in particular a protected peptide, or is at least one amino acid, natural or not, protected or not.

The term “peptide” designates a molecule constituted by at least two amino acids; said amino acids being bonded by an amide type chemical bond. A peptide derivative is a product which is chemically and structurally closely related to peptides, for example, pseudo peptides or peptide analogue products, peptide fragments, protected peptides (Sewald N., Jakubke H. D., peptides: chemistry and biology, Wiley CH, Weinheim, 2005, 5-55).

The term “peptide derivative” corresponds in particular to a protected peptide being protected on its N-terminus and/or on its C-terminus and/or on at least one of its side chains

According to another advantageous embodiment, the invention relates to the use of centrifugal partition chromatography in an ion exchange displacement mode such as here above defined, wherein said at least one displacer and said at least one retainer are cationic, and said at least one exchanger is anionic.

As described above, three distinct types of ion pairs are formed:
the pair between the at least one product to be purified and the exchanger,
the pair between the retainer and the exchanger, and
the pair between the displacer and the exchanger.

If the exchanger is anionic, then the displacer, the retainer and the product to be purified have to show at least a cationic moiety.

It is important to note that amphoteric products, because of their chemical nature, may be either cationic or anionic. Thus, in an ion exchange displacement mode, amphoteric products can be purified with either an anionic or cationic exchanger.

Ion exchange displacement mode CPC purification with a cationic displacer is called cationic mode. Ion exchange displacement mode CPC purification with an anionic displacer is called anionic mode.

In the present embodiment it has been found that cationic mode is advantageous.

According to another advantageous embodiment, the invention relates to the use of centrifugal partition chromatography in an ion exchange displacement mode, wherein several displacers and one exchanger are used.

The possibility to use several displacers and one exchanger allows the user to combine strong ion exchange and weak ion exchange modes. This association has never been carried out and allows the improvement of the selectivity between various peptides.

According to another advantageous embodiment, the invention relates to the use of centrifugal partition chromatography in an ion exchange displacement mode, wherein one displacer and one exchanger at various percentages of deprotonation are used, and said percentages of deprotonation varying from 1% to 100%, and in particular from 1% to 30%, and in particular from 5% to 50%.

Said percentages of deprotonation correspond to different activation rates of the exchanger and vary from 1% to 33%,

The possibility of dividing the column into zones corresponding to different activation rates of the exchanger leads to a new stationary phase design which is called “segmented stationary phase”. Such a segmented stationary phase has never been used in the CPC process. This segmented stationary phase which involves one displacer and the same exchanger at different activation degrees, is made possible because of the liquid nature of the stationary phase and enhances the selectivity between various peptides.

According to another advantageous embodiment, the invention relates to the use of centrifugal partition chromatography in an ion exchange displacement mode, wherein several displacers and one exchanger at various percentages of deprotonation are used.

The invention relates to a process for the purification of at least one product to be purified from a substrate by centrifugal partition chromatography in an ion exchange displacement mode, said product to be purified being an amphoteric product or an amphoteric convertible product,

wherein said process comprises:

at least one step of rotation of a centrifugal partition chromatography column, said column comprising a chromatographic mixture containing said substrate, a biphasic solvent mixture, at least one displacer, at least one exchanger and at least one retainer, said biphasic solvent mixture being constituted by two non-miscible phases, one phase being the stationary phase and the other phase being the mobile phase, and

at least one step of pumping of said aqueous mobile phase through said column, for a time sufficient to purify said product to be purified,

a step of recovery of said at least one product to be purified in a purified form.

Said “two non miscible phases” can be
an organic phase and an aqueous phase (in this case the organic phase is the stationary phase and the aqueous phase is the mobile phase),
an organic phase and a phase in which one at least of the solvent is miscible with water.

The solvent mixture is the purification media of the chromatography; this solvent mixture combination generates two distinct liquid phases, a stationary one and a mobile one.

The term “non-miscible phases” designates a solvent mixture which forms two phases for at least one proportion of the solvents that are present in the said solvent mixture. In other words, there is at least one proportion of the solvents such that the organic phase is not miscible within the aqueous phase and conversely.

CPC is a liquid-liquid purification technique, thus it requires two liquids in a biphasic system.

A liquid phase called the stationary phase constitutes the support that will interact with the products to be purified in order to slow their propagation through the column. The stationary phase remains within the column because of the gravitational field induced by the column rotation.

A liquid phase called the mobile phase is pumped through the column during the purification process.

During the purification process the volume of the two phases are at hydrodynamic equilibrium. For a biphasique solvent system, a centrifugal force field, a flow rate and a temperature, this equilibrium is defined by the ratio stationary phase/mobile phase allawing the percolation of the mobile phase through the stationary one without loss of the latter. The same amount of mobile phase pumped in the column at the input of the column is collected at the output of the column.

The amount of mobile phase that has to be pumped in the column depends on the interactions between the products to be purified and the exchanger in the stationary phase. If the product to be purified migrates slowly, a larger volume of mobile phase will be required, and the time sufficient for the purification of said product to be purified will be longer. On the contrary, if the product to be purified migrates quickly, a smaller volume of mobile phase will be required, and the time sufficient for the purification of said product to be purified will be shorter.

The man skilled in the art knows that a sufficient time for purification varies for each combination of parameters. A sufficient time for purification allows the recovery of the products that are purified.

The term “recovery” designates the collection of the product to be purified in its purified form at the output of the column. During the purification, the mobile phase pumped through the column is collected at the output of the column. The mobile phase collected may contain impurities, mixture of impurities, products to be purified in a purified form, mixtures of different products to be purified, or mixtures of impurities and product to be purified. The mobile phase is collected in different batches. The separation between two batches during the collection may be arbitrary or determined by a time scale, a volume, colour, detection apparatus (for example a UV spectrometer) or any other means.

The term “products to be purified in a purified form” designates at least one of the products in a form which is considered pure enough for the intended application. It encompasses pure product, that means product without a trace of any other product or impurities, or a mixture of products and/or impurities in amount that are satisfactory for the intended use of the said product to be purified in a purified form.

The rotation of the column is compulsory because the gravitational field generated induces the retention of the stationary phase when the mobile phase is pumped through the column.

It is important to note that the displacement mode still requires the use of a mobile phase which is pumped through the column. This flow of mobile phase may enable a separation based on elution mode. Thus, a run based on the displacement mode purification may comprise an elution mode part. This implication of the elution mode can be observed when one or several gaps (in other word a zone containing no product to be purified) between the different members of the isotachtic train appear. In an extreme case, when the elution mode part is important, each of the ion pairs formed due to the displacement mode are separated by a gap.

The combination of ion exchange displacement mode and elution mode can be particularly favourable, since it increases the resolution of the purification.

In the present invention, the substrate is soluble, or partially soluble, in water.

According to another advantageous embodiment, the invention relates to a process such as here above defined, wherein said substrate is soluble in water.

According to another advantageous embodiment, the invention relates to a process such as here above defined, wherein said substrate is partially soluble in water.

The terms “partially soluble” means that at least a small amount of the said substrate should be able to be solubilised in the aqueous phase.

It is important for the substrate to be soluble, or partially soluble, in water because in the preferred embodiments of the present invention, the mobile phase is an aqueous phase. If the substrate is not soluble in the aqueous phase (mobile phase), it will remain in the organic phase (stationary phase) and no purification will be possible.

The product to be purified should be at least to a small extend soluble in the mobile phase in order to be able to migrate through the column. Solubility between an organic and an aqueous phase can be measured by the partition coefficient.

The partition coefficient is the ratio of concentration of a product (solute) in the two phases of a mixture of two immiscible solvents at equilibrium. Hence, this coefficient is a measure of the differential solubility of the product between these two solvents, which are generally water and octanol. The logarithm of the ratio of the concentrations of the unionized solute in the solvents is called log P:

$$log {P}_{oct / wat} = log ( \frac{{[ solute ]}_{octanol}}{{[ solute ]}_{water}^{un - ionized}} )$$

The lower is the log P value of a given molecule, the more soluble in water the given molecule is.

The product to be purified is different from the “product to be purified—exchanger” ion pair.

As described above, the substrate is soluble, or partially soluble, in water. Thus substrate according to the present invention should have a low log P.

The “product to be purified—exchanger” ion pair is soluble in the organic phase. Thus product to be purified—exchanger” ion pair according to the present invention should have a high log P.

According to another advantageous embodiment, the invention relates to a process such as here above defined, wherein said substrate is hydrophobic.

The term “hydrophobic” characterises a product which has no affinity for water; or is tending to repel and not to absorb water; or is tending not to dissolve in water, or not to mix with water, or not to be wetted by water. Said hydrophobic products tend to have high log P values.

According to another advantageous embodiment, the invention relates to a process such as here above defined, wherein said substrate is a crude reaction mixture or a crude natural product extract from plant or biotechnological media.

The term “crude reaction mixture” designates a substrate which is a reaction mixture that has not been purified. This substrate may, or not, have been neutralized in order to stop the chemical reaction from which the said reaction mixture originates, or filtrated prior to the purification described in the present application.

The term “crude natural product extract” designates a substrate which is a raw material from biological source such as plants, bacteria, fungi, unicellular organisms, animals or any other kind of living cells, or even sediments containing organic substances. Said raw material may have been pre-treated or not prior to the purification described in the present application. Said pre-treatment includes but are limited to: filtration, grinding, extraction by a solvent (distillation, stem distillation, brew or any other technique).

The term “biotechnological media” designates a substrate which is obtained from living cells, enzymes, or ex-vivo biological systems. Said biotechnological media may have been filtrated, neutralized or partially purified by other chromatographic techniques prior to the purification described in the present application.

The invention relates to a process for the purification of at least one product to be purified from a substrate by centrifugal partition chromatography in an ion exchange displacement mode, said product to be purified being an amphoteric product, wherein said process comprises:

at least one step of rotation of a centrifugal partition chromatography column, said column comprising a chromatographic mixture containing said substrate, a biphasic solvent mixture, several displacers, one exchanger and at least one retainer, said biphasic solvent mixture being constituted by two non-miscible phases, one phase being the stationary phase and the other phase being the mobile phase, and

at least one step of pumping of said aqueous mobile phase through said column, for a time sufficient to purify said product to be purified,

a step of recovery of said at least one product to be purified in a purified form.

The invention relates to a process for the purification of at least one product to be purified from a substrate by centrifugal partition chromatography in an ion exchange displacement mode, said product to be purified being an amphoteric product, wherein said process comprises:

at least one step of rotation of a centrifugal partition chromatography column, said column comprising a chromatographic mixture containing said substrate, a biphasic solvent mixture, one displacer and one exchanger at various percentages of deprotonation are used, said percentages of deprotonation varying in particular from 1% to 30%. and at least one retainer,

said biphasic solvent mixture being constituted by two non-miscible phases, one phase being the stationary phase and the other phase being the mobile phase, and

at least one step of pumping of said aqueous mobile phase through said column, for a time sufficient to purify said product to be purified,

a step of recovery of said at least one product to be purified in a purified form.

The invention relates to a process for the purification of at least one product to be purified from a substrate by centrifugal partition chromatography in an ion exchange displacement mode, said product to be purified being an amphoteric product, wherein said process comprises:

at least one step of rotation of a centrifugal partition chromatography column, said column comprising a chromatographic mixture containing said substrate, a biphasic solvent mixture, several displacers and one exchanger, wherein one displacer and one exchanger at various percentages of deprotonation are used, said percentages of deprotonation varying in particular from 1% to 50%.

said biphasic solvent mixture being constituted by two non-miscible phases, one phase being the stationary phase and the other phase being the mobile phase, and

at least one step of pumping of said aqueous mobile phase through said column, for a time sufficient to purify said product to be purified,

a step of recovery of said at least one product to be purified in a purified form.

The present invention may be used for the purification of peptides or peptide derivatives, in particular protected peptides, or single amino acids, in particular protected single amino acids.

According to another advantageous embodiment, the invention relates to a process such as here above defined, wherein said at least one product to be purified contains less than about 100 amino acids, preferably less than about 80 amino acids, preferably less than about 60 amino acid residues, preferably less than about 50 amino acids, preferably less than about 40 amino acids, preferably less than about 20 amino acids, preferably from 80 to 20 amino acids, preferably from 80 to 50 amino acids, preferably from 50 to 20 amino acids.

Amino acids are molecules that have both a carboxylic acid function and an amine function. 22 amino acids are naturally occurring: Alanine (Ala), Arginine (Arg), Asparagine (Asn), Aspartic acid (Asp), Cysteine (Cys), Glutamic acid (Glu), Glutamine (Gln), Glycine (Gly), Histidine (His), Isoleucine (Ile), Leucine (Leu), Lysine (Lys), Methionine (Met), Phenylalanine (Phe), Proline (Pro), Pyrrolysine (Pyl), Seleno-cysteine (Sec), Serine (Ser), Threonine (Thr), Tryptophan (Trp), Tyrosine (Tyr), and Valine (Val). The amino acids can be natural but any other chemical product having both a carboxylic acid function and an amine function may be considered as an amino acid according to the present invention. Non-natural amino acids are not present in natural product extract.

According to another advantageous embodiment, the invention relates to a process such as here above defined, wherein said at least one product to be purified is a protein, and said protein contains from 1000 to 100 amino acids, preferably from 1000 to 300 amino acids, preferably from 500 to 100 amino acids, preferably from 1000 to 500 amino acids, preferably from 500 to 300 amino acids, preferably from 300 to 100 amino acids or wherein said at least one product to be purified is a peptide or is a peptide derivative, in particular a protected peptide, or is an amino acid, natural or not, protected or not.

The purification of proteins having more than 1000 aminoacids may be difficult because of the conditions of the CPC purification. The quaternary and tertiary structures of said proteins may be denatured by the contact with organic solvents.

According to another advantageous embodiment, the invention relates to a process such as here above defined, wherein said at least one product to be purified is an amino acid, natural or not, protected or not.

Protecting groups for amino acids may be found in books well known in the scientific literatures such as “protective groups in organic synthesis” (T. W. Greene and P. G. M. Wuts, 1999 John Wiley & Sons, Inc., ISBNs: 0-471-16019-9 (Hardback); 0-471-22057-4 (Electronic)). For example said protective groups may be: Boc, Fmoc, Bz, Bn, Aloc, trt, Pbf, tBu, ocHx, ODmp, StBu, Allyl, Hmb, OMe, OEt, Fm, Xan, Tmob, Mtt, Cbz, Npys, Acm, Dnp, Mis.

Polarity of the product to be purified is an important characteristic in the present invention. The polarity may be due to polar amino acids or functionalization of the product to be purified by polar moieties.

According to another advantageous embodiment, the invention relates to a process such as here above defined, wherein said at least one product to be purified contains less than about 80% polar amino acids, preferably less than about 70% polar amino acids, preferably less than about 60% polar amino acids, preferably less than 50% polar amino acids.

Polar amino acids according to the present invention are arginine, histidine, lysine, pyrrolysine, aspartic acid, glutamic acid, serine, threonine, asparagine, thyrosine, cysteine, selenocysteine and glutamine, as described in the book “Peptides from A to Z” by Hans-Dieter Jakubke and Norbert Sewald, pages 20-21 (2008, Wiley—VCH, ISBN 978-3-527-31722-6).

Peptides with more than 80% polar amino acids, with respect to the total number of amino acids, are difficult to extract from the aqueous phase (mobile phase). They may not interact properly with the exchanger (in the organic phase) and thus be difficult to purify.

According to another advantageous embodiment, the invention relates to a process such as here above defined, wherein said at least one product to be purified contains aminoacids functionalised by polar chemical moieties, such as carbohydrates, pegylated moieties, aliphatic chains, said aliphatic chains being functionalised or not, cyclic or not, branched or not, toxins for example:
Doxorubicin;
N-acetyl-g-calicheamicin
Maytansine derivatives;
Monomethyl auristatin derivatives;
CC-1065 and duocarmycib derivatives, including cyclopropabenzindole (CBI) analogs;
Irinotecan;
Taxane derivatives;
Saporin;
Epirubicin;
Clofaribine,

cytotoxic agents for example:
Mercaptopurine;
methotreate,
DNA, modified DNA i.e. DNA with modified bases or DNA with a modification of the phosphorus bonds, RNA, modified RNA i.e. RNA with modified bases or RNA with a modification of the phosphorus bonds, SiRNA, or modified SiRNA i.e. SiRNA with modified bases or SiRNA with a modification of the phosphorus bonds.

The term “polar chemical moieties” designates a chemical function having a dipolar moment superior to 0 Debye. This dipolar moment is induced by one or more heteroatom such as oxygen, nitrogen, sulphur, phosphorus, selenium, or halogen such as fluorine, chlorine, bromine, iodine.

The term “pegylated” designates an amino acid functionalised by a polyethylene glycol chain.

According to another advantageous embodiment, the invention relates to a process such as here above defined, wherein said at least one product to be purified is a peptide or peptide derivatives, in particular protected peptides containing less than about 40 amino acid residues, and less than 60% of said amino acid residues are polar amino acid residues.

According to another advantageous embodiment, the invention relates to a process such as here above defined, wherein said at least one product to be purified is a peptide of formula:

(SEQ ID NO: 1)
H-Asp-Glu-Asn-Pro-Val-Val-His-Phe-Phe-Lys-Asn-
Ile-Val-Thr-Pro-Arg-Thr-OH

According to another embodiment, the invention relates to a process such as here above defined, wherein said biphasic solvent mixture is biphasic and constituted by 2 to 5 different solvents, preferably 3 different solvents, particularly 4 different solvents.

According to another advantageous embodiment, the invention relates to a process such as here above defined, wherein water and n-butanol are two of the solvents constituting said biphasic solvent mixture.

According to another advantageous embodiment, the invention relates to a process such as here above defined, wherein one of the solvents contained in said biphasic solvent mixture is a solvent less polar than n-butanol, such as alkanes, ethyl acetate, chlorinated solvent, lipophilic esters, lipophilic ketones, lipophilic ethers.

Polarity of a product could be defined by two different parameters: the electric dipole moment, or the dielectric constant.

The electric dipole moment (μ) is a measure of the separation of positive and negative electrical charges in a system of charges, that is, a measure of the polarity of the charge system. This value is expressed in Debye (D), and 1 D corresponds to 3.33564×10⁻³⁰ C·m. The higher the D value, the more polar is the solvent. The n-butanol has an electrical dipole moment of 1.66 D.

The dielectric constant, or relative static permittivity, of a material under given conditions is a measure of the extent to which it concentrates electrostatic lines of flux. It is the ratio of the amount of stored electrical energy when a voltage is applied, relative to the permittivity of a vacuum. The relative static permittivity is the same as the relative permittivity evaluated for a frequency of zero. This value is noted ∈. The higher the ∈ value, the more polar is the solvent. The n-butanol has a dielectric constant value of 17.84.

According to another advantageous embodiment, the invention relates to a process such as here above defined, wherein one of the solvents contained in said biphasic solvent mixture is a solvent miscible with both H₂O and n-butanol, such as methanol, ethanol, propanol, acetonitrile, acetone.

Said solvent miscible with H₂O and n-butanol, is less polar than water, but more polar than n-butanol.

According to another advantageous embodiment, the invention relates to a process such as here above defined, wherein said biphasic solvent mixture contains the following four solvents:
water, and
a solvent miscible with both H₂O and n-butanol, such as methanol, ethanol, acetonitrile, acetone and
n-butanol, and
a solvent less polar than n-butanol, such as alkanes, ethyl acetate, chlorinated solvent, lipophilic esters, lipophilic ketones, lipophilic ethers.

This biphasic solvent mixture is advantageous because of its hydrodynamic behaviour in the column. The solvent mixture thus obtained has a satisfactory polarity. Moreover, the substrates show a good solubility in this solvent mixture. This solvent mixture allows a good selectivity in the purification process.

According to another advantageous embodiment, the invention relates to a process such as here above defined, wherein said biphasic solvent mixture contains the four following solvents: water, acetonitrile, n-butanol, and methyl-tertbutyl ether.

This biphasic solvent mixture is particularly advantageous.

According to another advantageous embodiment, the invention relates to a process such as here above defined, wherein the solvent mixture contains
from about 40% to about 60%, preferably from about 45% to about 55% of water, expressed in volume with respect to the total volume of the solvent mixture, and
from about 10% to about 40%, preferably from about 15% to about 30% of n-butanol, expressed in volume with respect to the total volume of the solvent mixture.

According to another advantageous embodiment, the invention relates to a process such as here above defined, wherein the solvent mixture contains
from about 1% to about 20%, preferably from about 5% to about 15% of said solvent miscible with both H₂O and n-butanol, to the total volume of the solvent mixture.

According to another advantageous embodiment, the invention relates to a process such as here above defined, wherein the solvent mixture contains
from about 10% to about 40%, preferably from about 15% to about 30% of said solvent less polar than n-butanol, to the total volume of the solvent mixture.

According to another embodiment, the invention relates to a process such as here above defined, wherein said at least one exchanger is anionic, and said retainer is cationic.

According to another advantageous embodiment, the invention relates to a process such as here above defined, wherein said at least one exchanger and said retainer forms an ion pair.

The retainer is the counter-ion of the exchanger at the beginning of the purification. The ion pair retainer—exchanger is the ion pair with the lowest free enthalpy of formation among the ion pairs that are formed during the purification.

According to another advantageous embodiment, the invention relates to a process such as here above defined, wherein said at least one exchanger is soluble in organic solvents.

In this preferred embodiment of the invention, the stationary phase is the organic phase, thus the exchanger has to be soluble in organic solvent in order to remain in the stationary phase. If the exchanger is soluble in the mobile phase (aqueous phase), this might cause a leakage of the exchanger from the column.

According to another advantageous embodiment, the invention relates to a process such as here above defined, wherein said at least one exchanger and said at least one product to be purified form a pair of ions between the anionic exchanger and the cationic moiety of the amphoteric product to be purified, and wherein said ion pair is called “exchanger—product to be purified” ion-pair.

The term “cationic moiety of the amphoteric product” designates the fragment of the amphoteric product positively charged. The ionic bond is created between the positive charge of the amphoteric product and the negative charge of the exchanger. Amphoteric products may carry a negative charge, or a positive charge, or both negative and positive charges (zwitterionic product), or no charges. Interaction of the cationic moiety (the positive charge) with the exchanger does not mean that this moiety is dissociated from the rest of the amphoteric product.

According to another advantageous embodiment, the invention relates to a process such as here above defined, wherein said substrate comprises more than one product to be purified, and wherein each of said product to be purified forms a distinct “exchanger—product to be purified” ion-pair, thus forming as many “exchanger—product to be purified” ion-pairs as products to be purified from the substrate.

In a particular embodiment the present invention relates to the purification of several products to be purified in a mixture, wherein each of the products to be purified has a chemical structure different from the other products to be purified. Thus, each ion pairs obtained between one of said products to be purified and an exchanger will be distinct, in other word different, from the other ion pairs formed.

Each of the ion pairs formed has an intrinsic free enthalpy of formation, and said free enthalpy of formation may be similar or different from the free enthalpy of formation of the other ion pairs formed, even if the products to be purified have a closely related chemical structure.

Products to be purified which have closely related structures may have different affinities for the exchanger.

According to another advantageous embodiment, the invention relates to a process such as here above defined, wherein said “exchanger—product to be purified” ion pairs are soluble in organic solvents.

It is important for the “exchanger—product to be purified” ion pairs to be soluble, or partially soluble, in organic solvent. In other words, it is important for the “exchanger—product to be purified” ion pairs to partition preferentially in the stationary organic phase, because in the preferred embodiments of the present invention, the stationary phase is an organic phase.

According to another advantageous embodiment, the invention relates to a process such as here above defined, wherein said chromatographic mixture contains exchangers which are at a different ionization state.

The term “different ionization state” relates to the electronic configuration of the exchanger molecule. An exchanger may have different ionization states, depending on the chemical function it carries. This difference in ionization state is characterized by the overall electrical charge of the exchanger molecule, +1, +2, +3, +4, +5, for cationic exchanger, or −1, −2, −3, −4, −5 for anionic exchanger.

According to another advantageous embodiment, the invention relates to a process such as here above defined, wherein said at least one exchanger is an alkylated phosphoric acid derivative, particularly DEHPA.

The term “DEHPA” designates the product bis(2-ethylhexyl)phosphoric acid.

According to another advantageous embodiment, the invention relates to a process such as here above defined, wherein the quantity of said at least one exchanger is calculated from the number of amino acid of said product to be purified, and the said exchanger and the amino acids are in a molar ratio from about 1 to about 10, particularly from 2 to 6, preferably from 4 to 6.

The displacer used in the present invention forms an ion pair with the exchanger. The displacer is soluble in the mobile phase (aqueous phase), whereas the “displacer—exchanger” ion pair is soluble in the stationary phase (organic phase)

According to another embodiment, the invention relates to a process such as here above defined, wherein said at least one displacer is cationic.

The displacer introduced in the CPC column is in the form of an ion pair with a counter ion. Providing in this embodiment the displacer is a cation, thus the said counter-ion is an anion.

According to another advantageous embodiment, the invention relates to a process such as here above defined, wherein said at least one displacer is soluble in water.

According to another advantageous embodiment, the invention relates to a process such as here above defined, wherein said at least one displacer and said at least one exchanger form a pair of ions between the anionic exchanger and the cationic displacer, and wherein said ion pair is called “displacer—exchanger” ion-pair.

According to another advantageous embodiment, the invention relates to a process such as here above defined, wherein said “displacer—exchanger” ion pairs are soluble in organic solvents.

The present invention refers to the use of one exchanger and one displacer, or several displacers and one exchanger, or one displacer and several exchangers, or several displacers and several exchangers.

According to another advantageous embodiment, the invention relates to a process such as here above defined, wherein the energy corresponding to the free enthalpy of formation of said “displacer—exchanger” ion pair is higher to the energy of the free enthalpy of formation of any of the said “exchanger—product to be purified” ion pairs.

In this embodiment, one displacer and one exchanger are used. The affinity of the displacer for the exchanger is higher than the affinity of any of the products to be purified for the exchanger.

The following embodiment describes the combinations of several displacers and one exchanger, several exchangers and one displacer, and several displacers and several exchangers. The affinity of the ions forming the “displacer—exchanger” pairs with respect to the product to be purified, may be considered from the point of view of the displacer or the exchanger.

According to another advantageous embodiment, the invention relates to a process such as here above defined, wherein said chromatographic mixture contains at least two different exchangers.

In a particular embodiment of the present invention, it is possible to use a combination of several exchangers, and one displacer. The uses of different exchangers increase the resolution of the purification because the number of ion pairs that are susceptible to be formed, with respect to purification with only one exchanger, is multiplied by the number of exchangers present in the column.

In this embodiment the affinity of the displacer to any of the exchanger is higher than the affinity of any of the products to be purified for any of the exchangers.

According to another advantageous embodiment, the invention relates to a process such as here above defined, wherein the energy corresponding to the free enthalpy of formation of each of the said “displacer—exchanger” ion pairs is higher than the energy of the free enthalpy of formation of at least one of the said “exchanger—product to be purified” ion pairs.

In this embodiment, one or several displacers and one or several exchangers are used. Each of the displacers that may be used has an affinity for an exchanger, selected among the different exchanger that may be present, which is higher than the affinity of any of the products to be purified for the selected exchanger.

In other word, introduction of a displacer in a column that contains several products to be purified, enables the purification of at least one of the said product to be purified, and may not enable the purification of the other products to be purified.

According to another advantageous embodiment, the invention relates to a process such as here above defined, wherein each exchanger has an affinity for at least one displacer, higher than its affinity for each of the said products to be purified,

wherein said affinity is proportional to the energy corresponding to the free enthalpy of formation of said “displacer—exchanger” ion pairs, or said “exchanger—product to be purified” ion pairs.

In this embodiment, one or several displacers and one or several exchangers are used. Each of the exchangers that may be used and displacer, selected among the different displacers that may be present, forms an “displacer—exchanger” ion pairs which has a free enthalpy of formation higher than the free enthalpy of formation of any of the “product to be purified—exchanger” ion pairs that may be form.

According to another advantageous embodiment, the invention relates to a process such as here above defined, wherein said displacer is chosen in the list comprising H⁺ and metallic salt such as: Ca²⁺, Fe²⁺, Mg²⁺, Fe³⁺, Mn²⁺, K⁺, Cu²⁺.

According to another advantageous embodiment, the invention relates to a process such as here above defined, wherein said exchanger and said displacer are in a molar ratio from about 1 to about 15, particularly from 2 to 15, preferably from 5 to 10.

According to another advantageous embodiment, the invention relates to a process such as here above defined, wherein said displacer and said products to be purified are in a molar ratio from about 2 to about 300, particularly from 2 to 100, preferably from 5 to 100.

According to another embodiment, the invention relates to a process such as here above defined, wherein

said biphasic solvent mixture contains the four following solvents: water, acetonitrile, n-butanol, and methyl t-butyl ether (MtBE), and

said at least one displacer is Ca²⁺, and

said at least one exchanger is DEHPA, and

said at least one product to be purified is a peptide, preferably H-Asp-Glu-Asn-Pro-Val-Val-His-Phe-Phe-Lys-Asn-Ile-Val-Thr-Pro-Arg-Thr-OH.

The amino acids constituting the peptide to be purified are represented by the three letter code, this code is detailed here above. The “H—” end corresponds to the amino end of the peptide, the “—OH” end corresponds to the acid end of the peptide.

According to another advantageous embodiment, the invention relates to a process such as here above defined, wherein said biphasic solvent mixture contains the four following solvents: water, acetonitrile, n-butanol, and MtBE, and

said at least one displacer is Ca²⁺.

Said Ca²⁺ ions may be introduced as a salt, wherein the counter ion is the Cl⁻ anion, thus in the form of a CaCl₂ salt.

Counter anions may be for example chloride, iodide, bromide, nitrate, acetate, citrate, sulphate, trifluoroacetate, formate, hydrogenophosphate, oxalate, and palmoate:

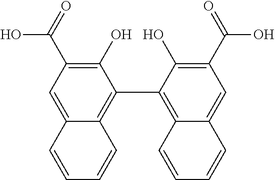

According to another advantageous embodiment, the invention relates to a process such as here above defined, wherein said biphasic solvent mixture contains the four following solvents: water, acetonitrile, n-butanol, and MtBE, and

said at least one exchanger is DEHPA.

According to another advantageous embodiment, the invention relates to a process such as here above defined, wherein said biphasic solvent mixture contains the four following solvents: water, acetonitrile, n-butanol, and MtBE, and

said at least one displacer is Ca²⁺, and

said at least one exchanger is DEHPA.

According to another advantageous embodiment, the invention relates to a process such as here above defined, wherein said biphasic solvent mixture contains the four following solvents: water, acetonitrile, n-butanol, and MtBE, and

said at least one product to be purified is a peptide, or peptide derivatives, in particular protected peptides, preferably H-Asp-Glu-Asn-Pro-Val-Val-His-Phe-Phe-Lys-Asn-Ile-Val-Thr-Pro-Arg-Thr-OH.

According to another advantageous embodiment, the invention relates to a process such as here above defined, wherein:

said at least one exchanger is DEHPA and

said at least one product to be purified is a peptide, or peptide derivatives, in particular protected peptides, preferably H-Asp-Glu-Asn-Pro-Val-Val-His-Phe-Phe-Lys-Asn-Ile-Val-Thr-Pro-Arg-Thr-OH.

According to another advantageous embodiment, the invention relates to a process such as here above defined, wherein:

said at least one displacer is Ca²⁺ and

said at least one product to be purified is a peptide, or peptide derivatives, in particular protected peptides, preferably H-Asp-Glu-Asn-Pro-Val-Val-His-Phe-Phe-Lys-Asn-Ile-Val-Thr-Pro-Arg-Thr-OH.

According to another advantageous embodiment, the invention relates to a process such as here above defined, wherein:

said at least one exchanger is DEHPA and

said at least one displacer is Ca²⁺ and

said at least one product to be purified is a peptide, or peptide derivatives, in particular protected peptides, preferably H-Asp-Glu-Asn-Pro-Val-Val-His-Phe-Phe-Lys-Asn-Ile-Val-Thr-Pro-Arg-Thr-OH.

According to another advantageous embodiment, the invention relates to a process such as here above defined, wherein:

said biphasic solvent mixture contains the four following solvents: water, acetonitrile, n-butanol, and MtBE, and

said displacer is Ca²⁺ and

said product to be purified is a peptide, or peptide derivatives, in particular protected peptides, preferably H-Asp-Glu-Asn-Pro-Val-Val-His-Phe-Phe-Lys-Asn-Ile-Val-Thr-Pro-Arg-Thr-OH.

According to another advantageous embodiment, the invention relates to a process such as here above defined wherein:

said biphasic solvent mixture contains the four following solvents: water, acetonitrile, n-butanol, and MtBE, and

said exchanger is DEHPA and

said product to be purified is a peptide, or peptide derivatives, in particular protected peptides, preferably H-Asp-Glu-Asn-Pro-Val-Val-His-Phe-Phe-Lys-Asn-Ile-Val-Thr-Pro-Arg-Thr-OH.

The invention relates to a process such as here above defined, comprising:
a step of introduction of the organic stationary phase in the Centrifugal Partition Chromatography column, said organic stationary phase comprising at least one exchanger and at least one retainer, and
a step of introduction of the substrate containing at least one product to be purified in the said Centrifugal Partition Chromatography column, and
a step of introduction of the aqueous mobile phase in the Centrifugal Partition Chromatography column, said mobile phase comprising at least one displacer, and
a step of pumping the said aqueous mobile phase through the said Centrifugal Partition Chromatography column, in order to enable the mobilization, of the said at least one product to be purified through the said column, and
a step of recovery of at least one of the said products to be purified in a purified form,
said column being in rotation from the introduction of the substrate to the recovery of at least one of the said products to be purified in a purified form.

A step of introduction of an aqueous mobile phase without displacer for a time sufficient to remove the products that do no form ion pairs with the exchanger may be added. This possible step occurs after the introduction of the substrate in the column, and prior to the introduction of the mobile phase containing the displacer.

The term “mobilization” refers to the capacity of the displacer contained in the mobile phase pumped through the column, to displace a “product to be purified—exchanger” ion pair in order to form a “displacer—exchanger” ion pair and a “product to be purified—counter ion” ion pair, which is then solubilised in the said mobile phase, and so move through the column toward the output with the flux of mobile phase.

The following embodiment describes a purification process and the state of the column at any moment while the purification process is running.

The invention relates to a process such as here above defined, comprising:
a step of introduction of the organic stationary phase in the Centrifugal Partition Chromatography column, said organic stationary phase comprising one exchanger, and at least one retainer, and
a step of introduction of the substrate containing at least one product to be purified in the said Centrifugal Partition Chromatography column, and
a step of introduction of the aqueous mobile phase in the Centrifugal Partition Chromatography column, said mobile phase comprises several displacers, and
a step of pumping the said aqueous mobile phase through the said Centrifugal Partition Chromatography column, in order to enable the mobilization of the said at least one product to be purified through the said column, and
a step of recovery of at least one of the said products to be purified in a purified form,
said column being in rotation from the introduction of the substrate to the recovery of at least one of the said products to be purified in a purified form.

The invention relates to a process such as here above defined, comprising:
a step of introduction of the organic stationary phase in the Centrifugal Partition Chromatography column, said organic stationary phase comprising one exchanger at various percentages of deprotonation, said percentages of deprotonation varying in particular from 1% to 50%, and at least one retainer, and
a step of introduction of the substrate containing at least one product to be purified in the said Centrifugal Partition Chromatography column and
a step of introduction of the aqueous mobile phase in the Centrifugal Partition Chromatography column, said mobile phase comprises at least one displacer, and
a step of pumping the said aqueous mobile phase through the said Centrifugal Partition Chromatography column, in order to enable the mobilization of the said at least one product to be purified through the said column, and
a step of recovery of at least one of the said products to be purified in a purified form,
said column being in rotation from the introduction of the substrate to the recovery of at least one of the said products to be purified in a purified form.

The invention relates to a process such as here above defined, comprising:
a step of introduction of the organic stationary phase in the Centrifugal Partition Chromatography column, said organic stationary phase comprising one exchanger at various percentages of deprotonation, said percentages of deprotonation varying in particular from 1% to 50%, and at least one retainer, and
a step of introduction of the substrate containing at least one product to be purified in the said Centrifugal Partition Chromatography column and
a step of introduction of the aqueous mobile phase in the Centrifugal Partition Chromatography column, said mobile phase comprises several displacers, and
a step of pumping the said aqueous mobile phase through the said Centrifugal Partition Chromatography column, in order to enable the mobilization of the said at least one product to be purified through the said column, and
a step of recovery of at least one of the said products to be purified in a purified form,
said column being in rotation from the introduction of the substrate to the recovery of at least one of the said products to be purified in a purified form.

The invention relates to a process such as here above defined, comprising:

at least one step of triggering the rotation of a Centrifugal Partition Chromatography column, said column comprising a separation mixture comprising said biphasic solvent mixture, said at least one retainer, said at least one exchanger, possibly said substrate, and possibly said at least one displacer,

wherein said centrifugal partition chromatography column comprises:
at one end, an input where both aqueous and organic phases, said retainer, said exchanger, possibly said substrate and possibly said displacer, are introduced in the column at an appropriate time, and
at the other end, an output where said organic stationary phase, said aqueous mobile phase, said retainer, said exchanger, possibly said products to be purified in a purified form, and possibly said displacer, are recovered from the column, and

wherein said displacement Centrifugal Partition Chromatography process allows the formation two to 2n+3 zones within the centrifugal partition chromatography column, and wherein n is the number of the different products to be purified from the substrate:
possibly a head zone, contiguous to the output of the column, said head zone comprising said retainer and said exchanger dissolved in said stationary phase,
possibly a tail zone, contiguous to the input of the column, wherein said tail zone comprises “displacer—exchanger” ion pairs,
central zones, situated between the head zone and the tail zone, or between the input and the output of the said column if respectively no tail zone or head zone are present,
   wherein the number of said central zones ranges from one to 2n+1,
   wherein
      the first central zone is the zone, among the central zones, which is the closest to the output of the column, or which is contiguous to the head zone if said head zone is present, and
      the last central zone, is the zone, among the central zones, which is the closest to the input of the column, or which is contiguous to the tail zone if said tail zone is present, and
   wherein, independently from each other, each of said central zones comprises or not, at least one “exchanger—product to be purified” ion pair, providing that at least one central zone comprises at least one product to be purified,
   preferably, at least one of the said product to be purified is located in a central zone containing no other product to be purified,
   preferably each of said n products to be purified is located in a central zone containing no other product to be purified.

Conditioning of the column is as follows:

The stationary phase (organic phase) and the exchanger—retainer pair are introduced prior to any other element, when the column is prepared for the purification. The displacer-free mobile phase (aqueous phase) is introduced after the column has been filled with stationary phase. The substrate is introduced prior to the displacer. The introduction of the mobile phase may occur before or after introduction of the substrate.

The term “zone” relates to different areas that may be observed in the column during the purification process. The relation between the displacement mode and the different zones is the mutual exclusion of the ion pairs which are formed within the column. In a theorical point of view, in each zone, the concentration of all the components involved in the separation process are constant and fixed by thermodynamic and acido-basic equilibrium.

As detailed above, three distinct types of ion pairs are formed:
the pair between the at least one product to be purified and the exchanger,
the pair between the retainer and the exchanger, and
the pair between the displacer and the exchanger.

The head zone corresponds to the cells of the column containing the pair between the retainer and the exchanger.

The central zones correspond to the cells of the column containing the pair between the product to be purified and the exchanger.

The tail zone corresponds to the cells of the column containing the pair between the displacer and the exchanger.

The number of zones constituting the central zones depends on the separation between the “product to be purified—exchanger” ion pairs, and on the presence or absence of elution mode

In purification by displacement without elution mode, the central zones are a set of one to a maximum of n different zones; wherein n is the number of the different products to be purified from the substrate. Each central zone corresponds to one or several specific “product to be purified—exchanger” ion pairs. One or more products to be purified may have the same retention time, which means that they migrate through the column at the same speed and are recovered at the output of the column together. These products are in the same central zone.

In other words, a single central zone corresponds to the absence of separation of the products; and n central zones correspond to the separation of each product to be purified from the other product to be purified.

The number of central zones is linked to the separation between the “product to be purified—exchanger” ion pairs, and is limited to a maximum corresponding to the maximum number of “product to be purified—exchanger” ion pairs that can be formed.

When the displacement mode is combined with elution mode, the mobile phase may create “gaps” between central zones containing product to be purified.

In an extreme case there is no effect of the elution, thus the number of central zones remains from one to n, as describe above.

In the opposite extreme case, the effect of the elution creates a gap between each central zone containing a product to be purified, thus 2n+1 zone are created. Central zones containing a product to be purified and “gap”, which contain no product to be purified, alternate.

The total number of zones includes the head zone containing the “retainer—exchanger” ion pair, and the tail zone containing the “displacer—exchanger” ion pair.

Thus, the total number of zone can reach a maximum up to 2n+3 zones, when
the purification process is running, and
the displacer is introduced and the retainer is not fully recovered from the column, and
each “product to be purified—exchanger” ion pairs are separated from the other “product to be purified—exchanger” ion pairs”, and
the elution mode creates gap between each zones (head, central and tail).

The minimum number of zones depends on different factors, in an extreme case the minimum zone can be one (retainer fully recovered, displacer not introduced, no separation by displacement or elution mode).

The wording “appropriate time” means that the aqueous and organic phases, retainer, exchanger, substrate displacer, are introduced at a time determined by the operator.

The following embodiment relates to the here above mentioned process, and further describes the mobile and stationary phases, the ions that they may contain, at any moment of the purification process and in any of the different zones.

According to another advantageous embodiment, the invention relates to a process such as here above defined, wherein said centrifugal partition chromatography column comprises
possibly a tail zone, contiguous to the input of the column, wherein
   said tail zone comprises a biphasic solvent mixture constituted by two non-miscible phases, namely the said stationary phase and the said mobile phase, and
   said tail zone comprises at least one displacer dissolved in the mobile phase, and at least one “exchanger—retainer” ion pair dissolved in the stationary phase, and
wherein, independently from each other, each of the central zones comprises or not, at least one “exchanger—product to be purified” ion pair dissolved in the stationary phase, and comprises or not, at least one product to be purified dissolved in the mobile phase,
   providing that at least one central zone comprises at least one product to be purified dissolved in the mobile phase, or at least one “exchanger—product to be purified” ion pair dissolved in the stationary phase,
   preferably, at least one of the said product to be purified, or at least one “exchanger—product to be purified” ion pair, is present in a central zone mobile phase containing no other product to be purified, or its respective central zone stationary phase a containing no other “exchanger—product to be purified” ion pair to be purified,
   preferably each of said n products to be purified, or “exchanger—product to be purified” ion pairs, is present in a central zone mobile phase containing no other product to be purified, or its respective central zone stationary phase containing no “exchanger—product to be purified” ion pair to be purified

Each of the zones here above defined comprises a mobile phase and a stationary phase. The purification process requires migration of the products to be purified between these two phases. However the ion pairs vary in function of the phase considered.

The ion pairs with the exchanger are formed in the stationary phase because the exchanger is not soluble in the mobile phase. Thus, the products to be purified present in the stationary phase form ion pairs with the exchanger. The formation of an ion pair in organic stationary phase, which stabilizes the products to be purified and consequently prevents their deterioration, and enables the recovery of said products in aqueous phase, also leads to a better preservation of the products to be purified and therefore enables the biological integrity of said products.

The mobile phase contains counter ions forming pairs with respectively the products to be purified, the retainer and the displacer. These counter ions are more soluble in the mobile phase than in the stationary phase. These counter ions correspond to the counter ions of the products to be purified or displacer introduced in the column.

Thus, the products to be purified are in the mobile phase form ion pair with a cation soluble in the mobile phase.

The mobile phase and the stationary phase are always electrically neutral. Each migration of anion or cation from one phase to the other is always balanced by a flux of electrically equivalent ion in the opposite direction.
According to another advantageous embodiment, the invention relates to a process such as here above defined,

wherein in said displacement Centrifugal Partition Chromatography process allowing the formation of two to 2n+3 zones within the centrifugal partition chromatography column, each zone corresponds to a respective batch which is recovered at the output of the column:
a head batch comprising the exchanger and the retainer dissolved in the organic stationary phase,
a tail batch comprising the displacer dissolved in the aqueous mobile phase,
central batches, situated between the head batch and the tail batch,
   wherein the number of said central batches ranges from 1 to 2n+1,
   wherein
      the first central batch is the batch, among the central batches, which is the first, chronologically, to be recovered at the output of the column, and
      the last central batch, is the batch, among the central batches, which is the last, chronologically, to be recovered at the output of the column, and
   wherein
      each of the central batches comprise aqueous mobile phase, and
      independently form each other, each of said central batches comprises or not, at least one product to be purified, and
      providing that at least one central batch comprises at least one product to be purified,
      preferably, at least one of the said product to be purified is located in a central batch containing no other product to be purified,
      preferably each of said n products to be purified is located in a central batch containing no other product to be purified.

Each of said central batch comprises, or not, one or more products to be purified. When more than one product be purified are present in the same central batch, it indicates that the said products to be purified have similar, or very close retention time, and thus migrate at the same speed through the column. This similar retention time may be due to “product to be purified—exchanger” ion pairs which have similar or close free enthalpies of formation. In other words, these products to be purified have similar chromatographic behaviour.

The purpose of purification is to isolate pure products or at least to remove products that are considered to be impurities from a mixture. The products in their purified form are recovered at the output of the column. Recovery is a continuous process since the displacer is continuously introduced in the column.

The term “continuous introduction” means that the mobile phase, possibly containing a displacer, is pumped into the column, at the input of the column at a defined rate, at that the pumping is regular and uninterrupted until the recovery of the product to be purified is considered to be achieved and the purification process is stopped.

It is important to keep in mind that the driving force of the emergence of the product at the output of the column, in displacement mode, is the introduction of an increasing amount of displacer in the column. The increasing amount of displacer literally “pushes” the products to be purified forming ion pairs with the exchanger, out of the column.

The term “continuous recovery” means that the mobile phase emerging from the column at the output of the column is recovered at a rate corresponding to the introduction rate of the mobile phase at the input of the column. Since the input is continuous and uninterrupted, and the column volume is fixed, the recovery at the output of the column is continuous and uninterrupted. Recovery of the mobile phase at the output of the column stops when the introduction of the mobile phase at the input of the column stops.

Leakage of the stationary phase may be observed during the purification process. The term “leakage” designates the recovery of stationary phase at the output of the column. Stationary phase is not supposed to be recovered during the purification, and should remain within the column. Leakage of the stationary phase is due to particular hydrodynamic behaviour of the two phases. The physico-chemical properties of the two phases (viscosity, interfacial tension, volumic mass difference) lead to driving phenomena of the stationary phase by the mobile one.

The following embodiment relates to a process starting from the beginning of the purification process, when no displacer has been introduced in the CPC column. The substrate has already been introduced and an organisation between the “products to be purified—exchanger” ions pairs based on affinity appears, but no displacement mode is processing.

According to said embodiment, the invention relates to a process such as here above defined,

wherein said displacement Centrifugal Partition Chromatography process allows the formation of two to 2n+2 zones within the centrifugal partition chromatography column, and wherein n is the number of the different products to be purified from the substrate, said zones consisting in:
a head zone, contiguous to the output of the column, said head zone comprising said retainer and said exchanger dissolved in said stationary phase, and
central zones, situated between the head zone and the output of the said column, wherein
   the number of said central zones ranges from 1 to 2n+1,
   the first central zone is the zone, among the central zones, which is the closest to the output of the column, and
   the last central zone, is the zone, among the central zones, which is the closest to the input of the column,

The following embodiment relates to the purification process of the invention, when the displacer has been introduced in the CPC column. The isotachtic train is formed and processes through the column toward the output of the column, while the displacer is continuously injected at the input of the column.

According to another embodiment, the invention relates to a process such as here above defined,

wherein said displacement Centrifugal Partition Chromatography process allows the formation of n+2 to 2n+3 zones within the centrifugal partition chromatography column, and wherein n is the number of the different products to be purified from the substrate, said zones consisting in:
a head zone, contiguous to the output of the column, said head zone comprising said retainer and said exchanger dissolved in said stationary phase, and
a tail zone, contiguous to the input of the column, wherein said tail zone comprises “displacer—exchanger” ion pairs, and
central zones, situated between the head zone and the tail zone,
   wherein
      the number of said central zones ranges from 1 to 2n+1,
      the first central zone is the zone, among the central zones, which is the closest to the output of the column, and
      the last central zone, is the zone, among the central zones, which is the closest to the input of the column.

The following embodiment relates to the purification process of the invention, when the purification is almost complete. The products to be purified have been fully recovered at the output of the column, except for one which is being recovered. The displacer is continuously injected at the input of the column.

According to said advantageous embodiment, the invention relates to a process such as here above defined,

wherein said displacement Centrifugal Partition Chromatography process allows the formation of n+1 to 2n+3 zones within the centrifugal partition chromatography column, and wherein n is the number of the different products to be purified from the substrate, said zones consisting in:
a tail zone, contiguous to the input of the column, wherein said tail zone comprises “displacer—exchanger” ion pairs, and
central zones, situated between the output of said centrifugal partition chromatography column and the tail zone, the number of said central zones ranges from 1 to 2n+1.

In this embodiment, the head zone comprising said retainer has already been fully recovered from the column, and thus is not considered.

The following embodiment relates to the purification process of the invention, wherein the specific purification conditions are specified.

According to said embodiment, the invention relates to a process such as here above defined, comprising:
a step of introduction of the organic stationary phase in the Centrifugal Partition Chromatography column, said organic stationary phase comprising at least one exchanger and at least one retainer, and said organic phase comprising the following four solvents:
   water less than 15% of the volume of the organic phase
   a solvent miscible with both H₂O and n-butanol, such as methanol, ethanol, acetonitrile, acetone at a volumic proportion less than 50%, and
   n-butanol (5 to 90%, v/v), and
   a solvent less polar than n-butanol (5 to 90% v/v), such as alkanes, ethyl acetate, chlorinated solvent, lipophilic esters, lipophilic ketones, lipophilic ethers, and
a step of introduction of the substrate comprising at least one product to be purified in the said Centrifugal Partition Chromatography column, and
a step of continuous introduction of the aqueous mobile phase in the Centrifugal Partition Chromatography column, said aqueous mobile phase comprising the following three solvents:
   water (more than 50%, v/v), and
   a solvent miscible with both H₂O and n-butanol at a volumic proportion less than 50%, such as methanol, ethanol, acetonitrile, acetone or a mixture thereof and
   possibly n-butanol less than 25%, and
   a solvent less polar than n-butanol less than 25%, such as alkanes, ethyl acetate, chlorinated solvent, lipophilic esters, lipophilic ketones, lipophilic ethers,
a step of continuous introduction of a least one displacer in said aqueous mobile phase, and pumping the aqueous mobile phase comprising the at least one displacer through the said Centrifugal Partition Chromatography column, in order to enable the mobilization of the said at least one product to be purified through the said column, and
a step of continuous recovery of at least one of the said products to be purified in a purified form, at the output of the column,
   wherein the continuous rotation of the column, the continuous introduction of a least one displacer in said aqueous phase, and the continuous pumping of said aqueous phase through the column, are maintained,
   said step of continuous recovery being triggered when the first central batch is recovered at the output of the column.

According to another advantageous embodiment, the invention relates to a process such as here above defined, wherein

said organic phase comprises acetonitrile, n-butanol, MtBE, and traces of water said aqueous mobile phase comprises water, acetonitrile, n-butanol, and traces of MtBE.

According to another embodiment, the invention relates to a process such as here above defined, comprising:
a step of introduction of the organic stationary phase in the Centrifugal Partition Chromatography column, said organic stationary phase comprising at least one exchanger and at least one retainer, and said organic phase comprising: acetonitrile, n-butanol, MtBE, and traces of water (less than 10%)
a step of introduction of the substrate comprising a peptide or a peptide derivative to be purified, in particular the SF 328 to be purified in the said Centrifugal Partition Chromatography column, and
a step of continuous introduction of the aqueous mobile phase in the Centrifugal Partition Chromatography column, and said aqueous mobile phase comprising water, acetonitrile, n-butanol, and traces of MtBE (less than 10%).
a step of continuous introduction of a least one displacer in said aqueous mobile phase, and pumping the aqueous mobile phase comprising the at least one displacer through the said Centrifugal Partition Chromatography column, in order to enable the mobilization of the said at least one product to be purified through the said column, and
a step of continuous recovery of the peptide or the peptide derivative to be purified, in particular the SF 328 to be purified in a purified form, at the output of the column,
   wherein the continuous rotation of the column, the continuous introduction of a least one displacer in said aqueous phase, and the continuous pumping of said aqueous phase through the column, are maintained,
   said step of continuous recovery being triggered when the first central batch is recovered at the output of the column.

According to another embodiment, the invention relates to a process such as here above defined, wherein

the step of continuous introduction of the aqueous mobile phase in the Centrifugal Partition Chromatography column, and the step of continuous introduction of a least one displacer in said aqueous phase, are repeated from 2 to 4 cycles,

said cycles being in a number sufficient to recover all the products to be purified,

wherein, for a given cycle, the displacer used is different from the displacer used in the previous cycles, and enables the formation of a “retainer—exchanger” ion pair with an affinity higher than the affinity of at least one of the “exchanger—product to be purified” ion pairs that remain in the column,

in order to enable, for each cycle, the recovery at the output of the column of at least one batch comprising at least one of the said product to be purified, remaining in the column, in a purified from.

The term “cycle” refers to a time period of a purification process; during said period at least one of the products to be purified is mobilized through the column and recovered at the output of the column. Said period starts at the introduction of a displacer in the column and ends at the recovery of the said displacer at the output of the column. In other words, a cycle starts when a mobile phase containing a displacer is introduced at the input of the column and ends when the product to be purified (in the mobile phase) is fully recovered at the output of the column.

When a cycle is over, that is to say no more products to be purified emerges at the output of the column, or in other words when the displacer emerges at the output of the column, a new cycle can be triggered by using a different displacer from the one previously used.

Prior to any cycle, the column is filled with the stationary phase containing the “exchanger—retainer” ion pair, the substrate is introduced in the column and the rotation of the column is triggered.

The use of several different displacers in distinct cycle is advantageous since the free enthalpy of formation of the “displacer—exchanger” ion pair may be tuned in function of the desired separation, that is the isolation or the partial purification of one product, or of several products.

For example, a displacer having an affinity to the exchanger that is weaker than the affinity of some of the products to be purified may be used. In this case, only a part of the products to be purified would be affected by the introduction of the displacer (the product to be purified with an affinity to the exchanger lower than the affinity of the introduced displacer).

By introduction of several displacers which each have an affinity for the exchanger higher than the previously introduced displacer, a gradient can be created, the force of the displacement mode being increased in a step wise manner for each cycle. This may provide an improved purification of mixtures of products to be purified.

According to another advantageous embodiment, the invention relates to a process such as here above defined, comprising:
a step of introduction of the organic stationary phase in the Centrifugal Partition Chromatography column, said organic stationary phase comprising at least one exchanger and at least one retainer, and said organic phase comprising: acetonitrile, n-butanol, MtBE, and traces of water (less than 10%) and
a step of introduction of the substrate containing at least one product to be purified in the said Centrifugal Partition Chromatography column, and
a step of continuous introduction of the aqueous mobile phase in the Centrifugal Partition Chromatography column, and said aqueous mobile phase comprising water and acetonitrile, n-butanol, and traces of MtBE (less than 10%), and
a step of continuous introduction of a least one displacer in said aqueous mobile phase, and pumping the aqueous mobile phase comprising the at least one displacer through the said Centrifugal Partition Chromatography column, in order to enable the mobilization of the said at least one product to be purified through the said column, and

said step of continuous introduction of the aqueous mobile phase in the Centrifugal Partition Chromatography column and said step of continuous introduction of a least one displacer in said aqueous phase steps

being repeated from 2 to 4 cycles, said cycles being in a number sufficient to recover all the products to be purified,

wherein, for a given cycle, the displacer used is different from the displacer used in the previous cycles, and enables the formation of a “retainer—exchanger” ion pair with an affinity higher than the affinity of at least one of the “exchanger—product to be purified” ion pairs that remain in the column,

in order to enable, for each cycle, the recovery at the output of the column of at least one batch comprising at least one of the said product to be purified, remaining in the column, in a purified from.

### DESCRIPTION OF THE FIGURES

FIG. 1 is a scheme representing the displacement process occurring within the CPC column when the substrate is introduced. It represents the sequence occurring before the displacer is introduced into the column, and thus before any purification by ion exchange displacement mode.

The figure is divided in a four time sequence, numbered from 0 to 3, starting from t=0, which represents the moment before the substrate is introduced into the CPC column. Time units are arbitrary and do not reflect any kind of reality. Time units are used to characterize different successive time sequences, in order to understand the displacement process.

The column is represented by a chain of six cells (several thousands in real CPC column), linked from the bottom of one cell to the top of the next cell. The black arrow represents the centrifugal field applied to the cells by rotation of the CPC column. The white arrow represents the direction of the pumping of the mobile phase, from the input of the column toward the output of the column.

The upper parts of the cells, which are colored in grey, represent the stationary phase. The lower parts of the cells, which are uncolored, represent the mobile phase.

The retainer is represented by a white disk.

In the present example the substrate is considered to contain three different products to be purified. The products to be purified from the substrate are represented by three triangles,

the black triangle represents the product to be purified which has a strong affinity for the exchanger,

the grey triangle represents the product to be purified which has a medium affinity for the exchanger, and

the white triangle represents the product to be purified which has a low affinity for the exchanger.

The exchanger is represented by a white square.

The counter ions are represented by a white star.

At t=0, each cell of the CPC column is filled with stationary and mobile phases in equilibrium. “Retainer—exchanger” ion pairs are solubilized in the stationary phase.

At t=1, the products to be purified are introduced into the column. The product to be purified with the high affinity for the exchanger forms an ion pair with the exchanger. The retainer is salted-out of the stationary phase into the mobile phase, and forms an ion pair with the counter ion of the product to be purified with the high affinity for the exchanger.

The products to be purified with medium and low affinity for the exchanger are not solubilized in the stationary phase because they cannot form an ion pair with the exchanger. They remain in the mobile phase and are eluted to the next cell.

At t=2, the product to be purified with the medium affinity for the exchanger form an ion pair with the exchanger in the second cell. Retainer is salted-out of the stationary phase into the mobile phase, and forms an ion pair with the counter ion of the product to be purified with the medium affinity for the exchanger.

The product to be purified with a low affinity for the exchanger is not solubilized in the stationary phase because it cannot form an ion pair with the exchanger. It remains in the mobile phase and is eluted to the next cell.

At t=3, the product to be purified with the low affinity for the exchanger forms an ion pair with the exchanger in the third cell. The retainer is salted-out of the stationary phase into the mobile phase, and forms an ion pair with the counter ion of the product to be purified with the low affinity for the exchanger.

All the products to be purified form ion pairs with exchangers, but in different cells because of their different affinity to the exchanger.

Mobile phase only contains “retainer—counter ion” ion pairs, which are not soluble in the stationary phase and are eluted towards the output of the column.

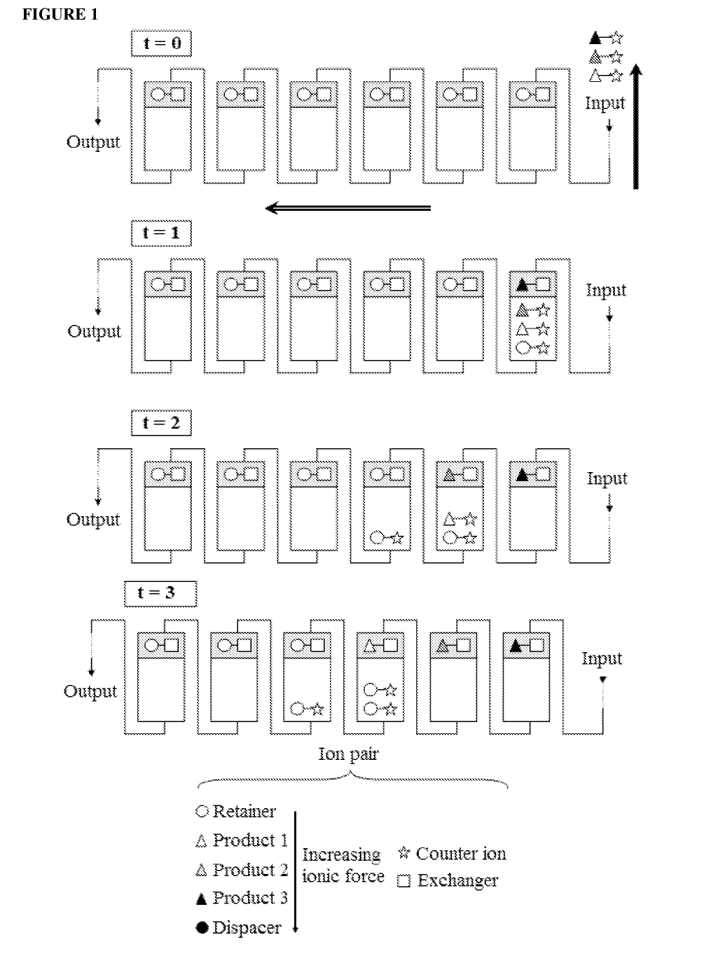

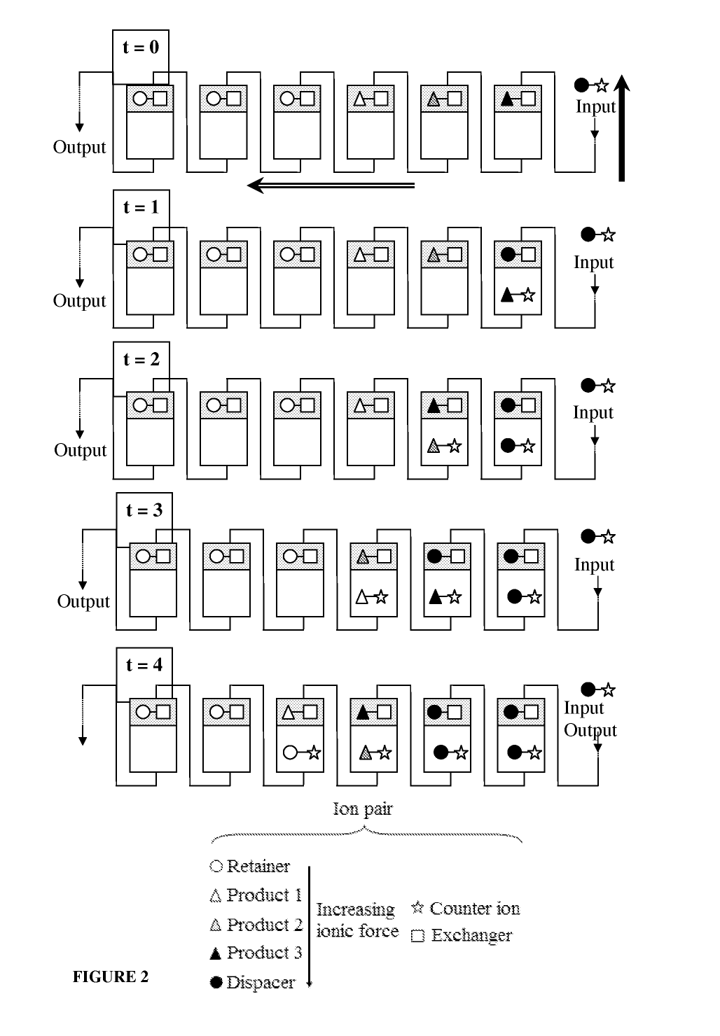

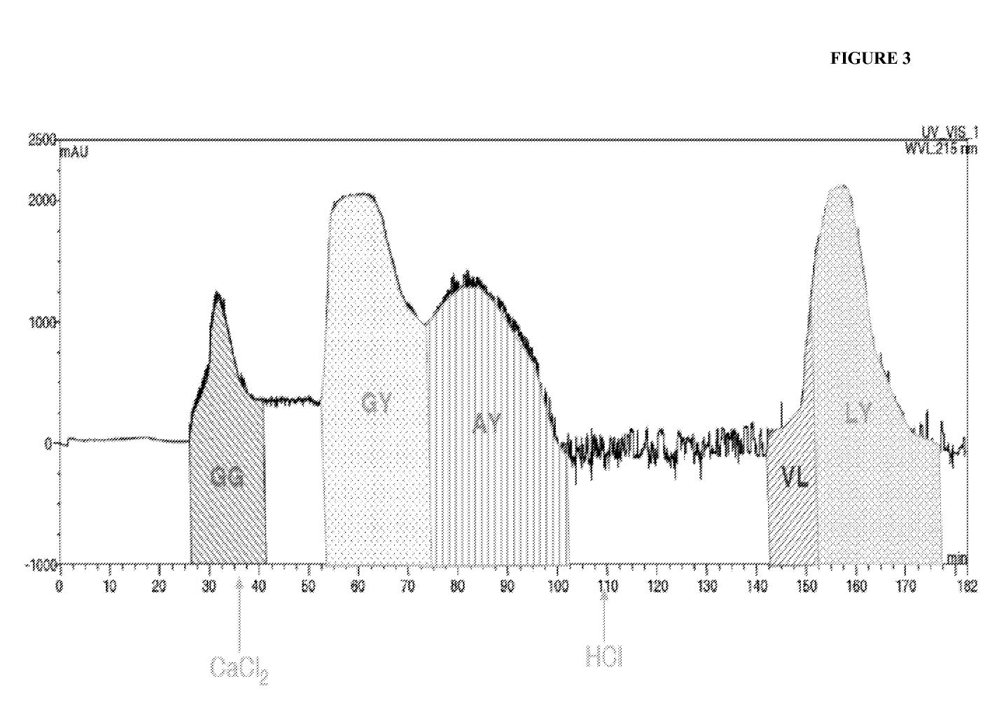

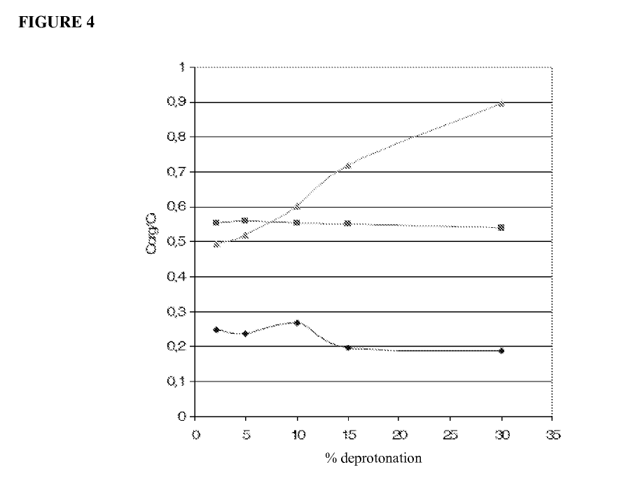

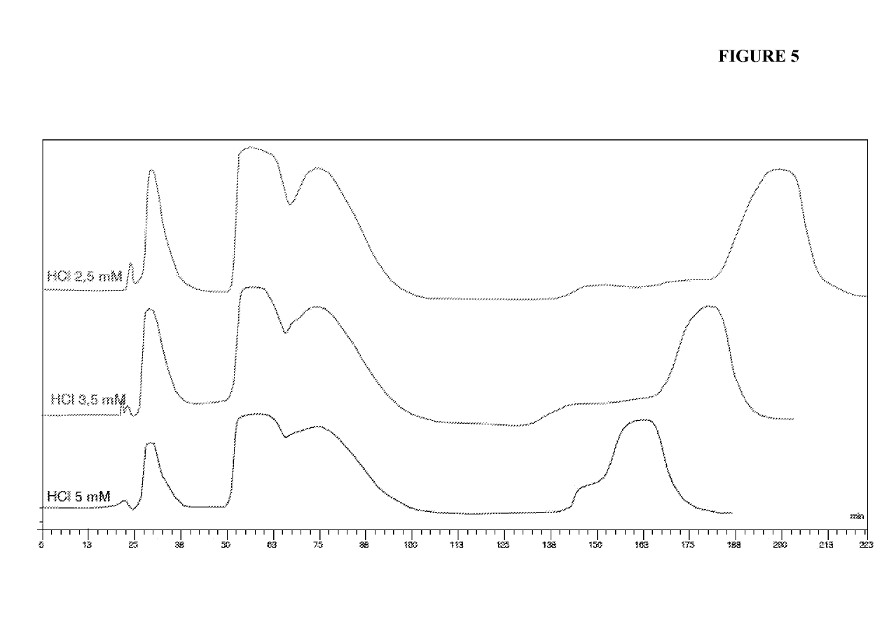

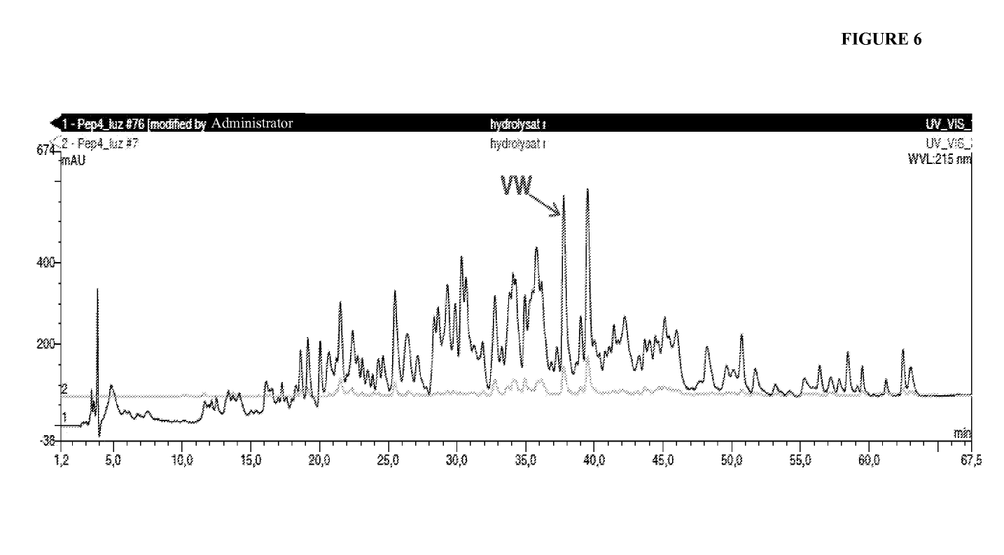

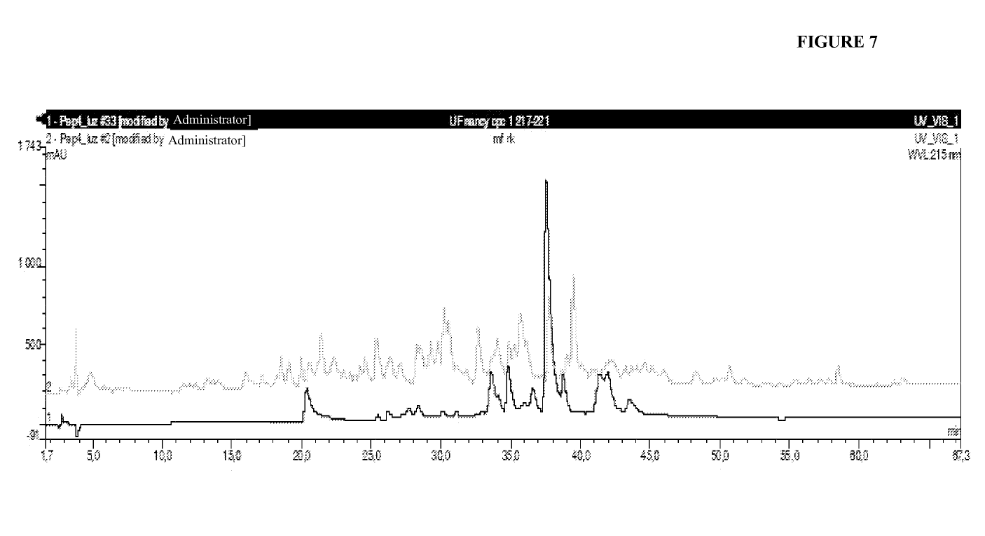

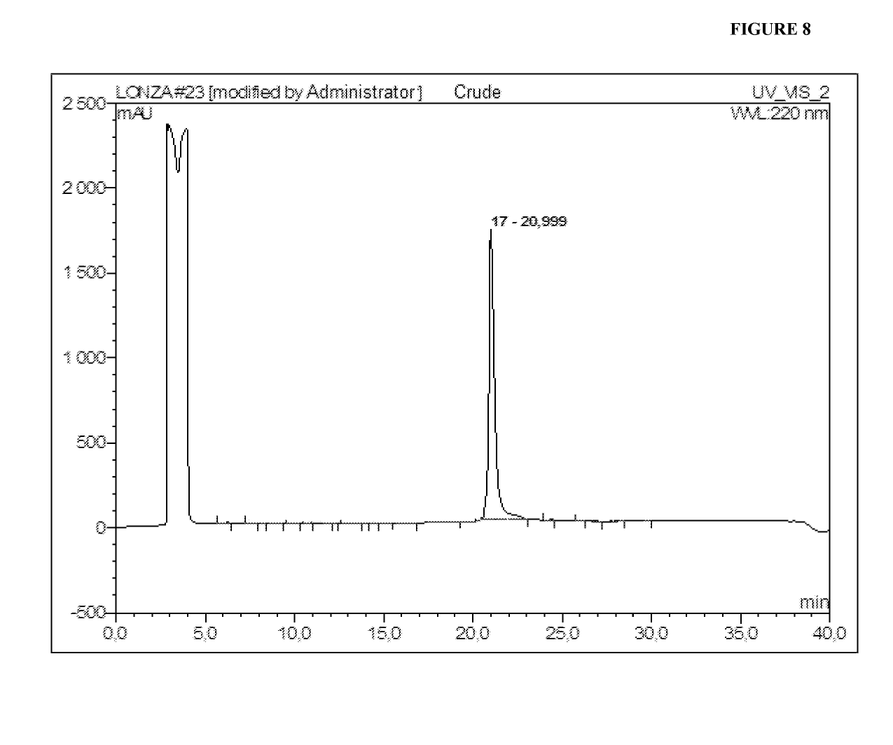

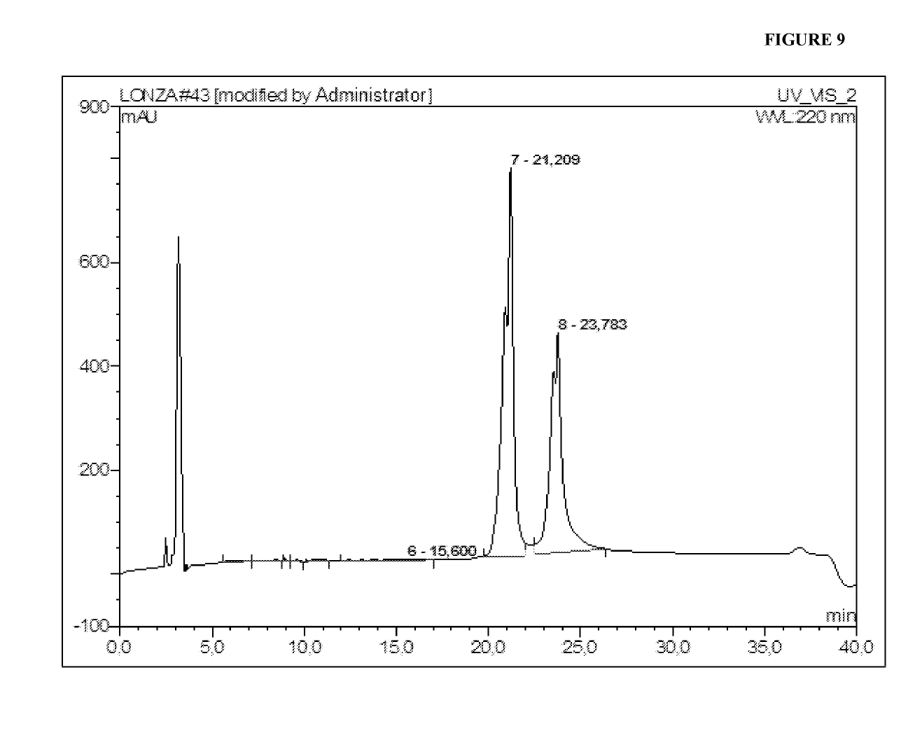

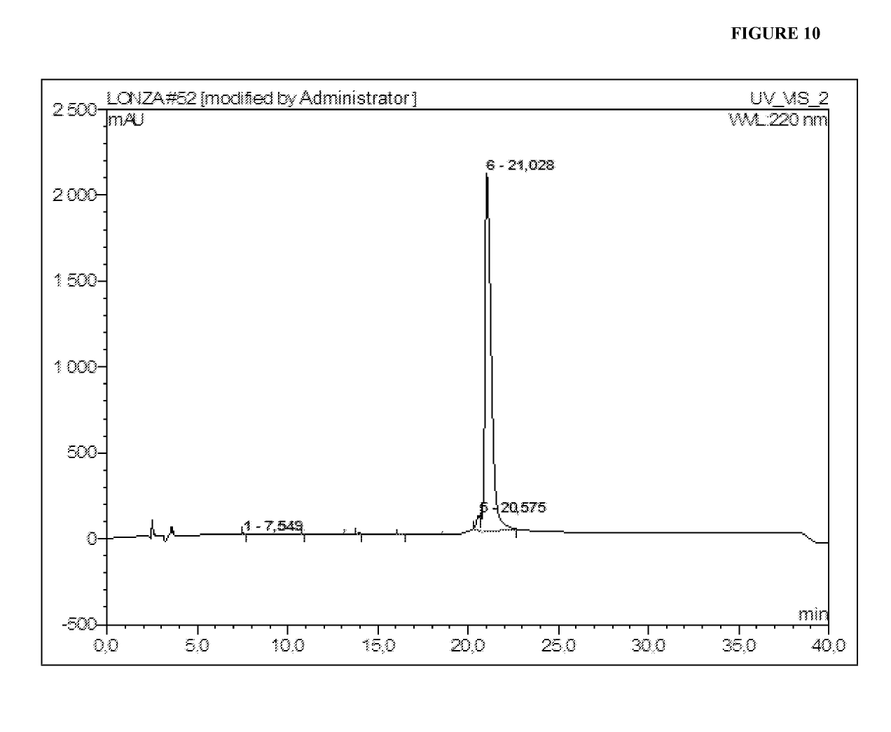

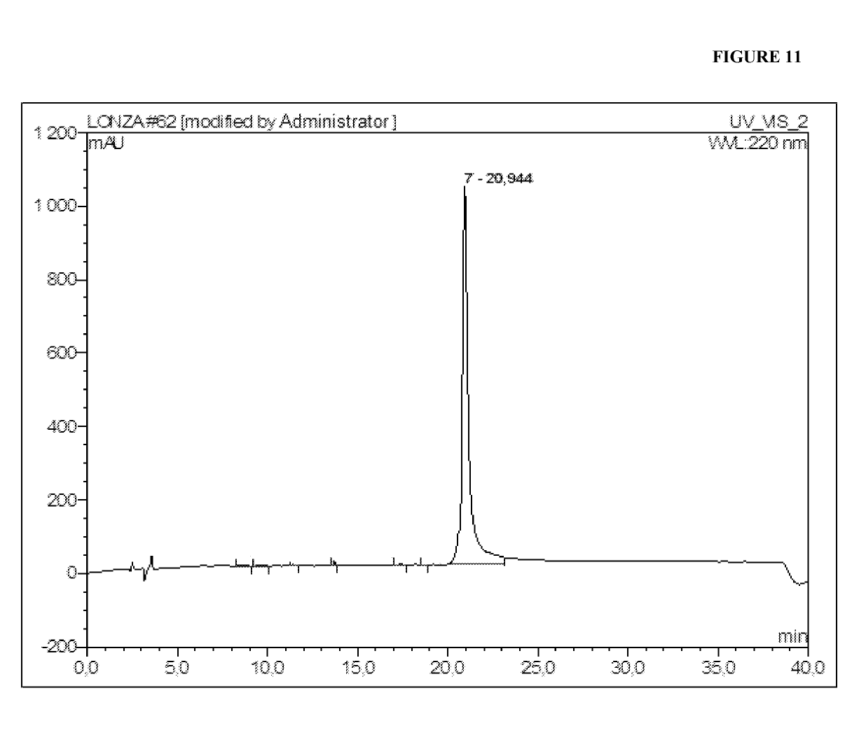

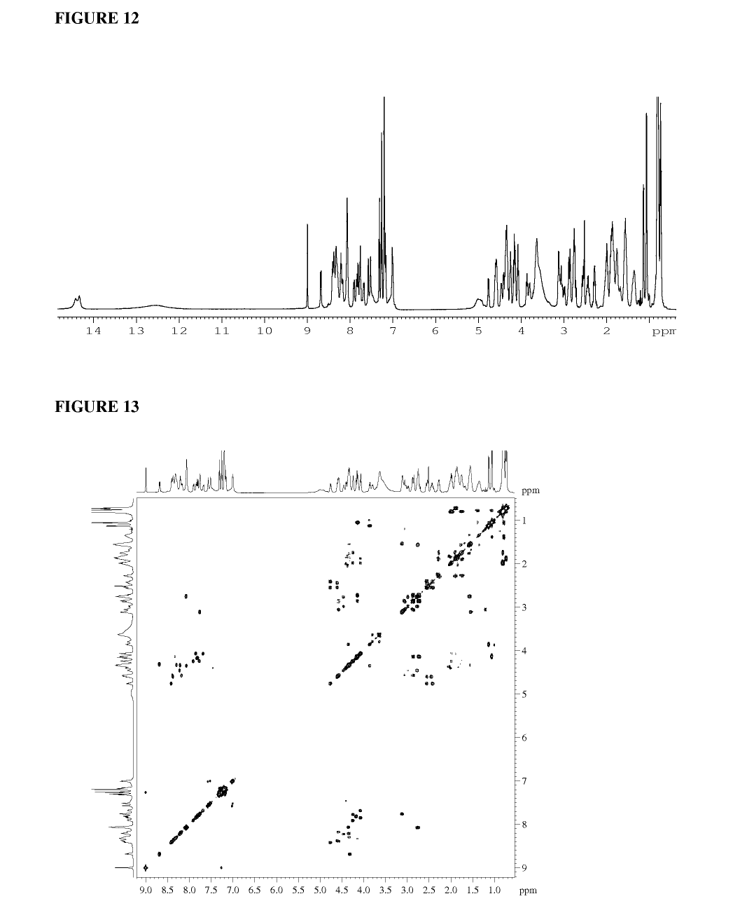

FIG. 2 is a scheme representing the displacement process occurring within the CPC column when the displacer is introduced. It represents the way the products to be purified, which have been introduced into the column as described in FIG. 1, are purified by ion exchange displacement mode. The process described in FIG. 2 occurs after the introduction of the products to be purified as described in FIG. 1.

The figure is divided in a five time sequence, numbered from 0 to 4, evolving from t=0, which represents the moment before the displacer is introduced into the CPC column.

Time unit, column representation, and symbols used are as described in FIG. 1.

The displacer is represented by a black disk.

The displacer is continuously introduced during the purification by displacement mode. Thus, for each time sequence, the displacer is introduced into the column at the input of the column.

At t=0, each cell of the CPC column is filled with stationary and mobile phases in equilibrium. “Product to be purified—exchanger” ion pairs are present in the cells next to the input of the column. The product, among all the products to be purified, having the strongest affinity for the exchanger is in the first cell from the input. The product, among all the products to be purified, having the lowest affinity for the exchanger is in the last cell from the input containing a “product to be purified—exchanger” ion pair.

The other cells contain “retainer—exchanger” ion pairs in the stationary phase.

This moment corresponds to the time t=3 of FIG. 1.

At t=1, the displacer is introduced into the column. The displacer forms an ion pair with the exchanger. The product, among all the products to be purified, having the strongest affinity for the exchanger is salted-out in the mobile phase, forms an ion pair with the counter ion of the displacer, and is eluted to the next cell.

At t=2, the displacer is introduced into the column. The stationary phase of the first cell from the input already contains a “displacer—exchanger” ion pair, thus the displacer newly introduced is not solubilized in the stationary phase and remains in the mobile phase and is eluted to the next cell.

The product, among all the products to be purified, having the strongest affinity for the exchanger is solubilized in the stationary phase of the second cell from the input. The product, among all the products to be purified, having a medium affinity for the exchanger is salted out into the mobile phase and forms an ion pair with the counter ion of the product (among all the products to be purified) having the strongest affinity for the exchanger. This ion pair elutes to the next cell.

At t=3 the displacer is introduced into the column. The stationary phases of the first and second cells from the input contain a “displacer—exchanger” ion pair. The mobile phase of the first cell contains a “displacer counter ion” ion pair.

The product, among all the products to be purified, having the strongest affinity for the exchanger is salted-out in the mobile phase of the second cell from the input, forms an ion pair with the counter ion of the displacer, and is eluted to the next cell.

The product, among all the products to be purified, having a medium affinity for the exchanger is solubilized in the stationary phase of the third cell from the input, and forms an ion pair with the exchanger.

The product, among all the products to be purified, having the lowest affinity for the exchanger is salted-out in the mobile phase of the third cell from the input, forms an ion pair with the counter ion of the product (among all the products to be purified) having a medium affinity for the exchanger, and is eluted to the next cell.

At t=4 the displacer is introduced into the column. The stationary phases of the first and second cells from the input contain a “displacer—exchanger” ion pair. The mobile phases of the first and second cells contain a “displacer counter ion” ion pair.

The product, among all the products to be purified, having the strongest affinity for the exchanger is solubilized in the stationary phase of the third cell from the input, and forms an ion pair with the exchanger.

The product, among all the products to be purified, having a medium affinity for the exchanger is salted-out in the mobile phase of the second cell from the input, forms an ion pair with the counter ion of the product (among all the products to be purified) having the strongest affinity for the exchanger, and is eluted to the next cell.

The product, among all the products to be purified, having the lowest affinity for the exchanger is solubilized in the stationary phase of the fourth cell from the input, and forms an ion pair with the exchanger.

The retainer contained in the stationary phase of the fourth cell from the input is salted out into the mobile phase, and forms an ion pair with the counter ion of the product (among all the products to be purified) having the lowest affinity for the exchanger. This “retainer—counter ion” ion pair is eluted toward the output of the column.

FIG. 3 is the elution profile of the compounds recorded by detection UV at 215 nm, at the output of a CPC column. In abscissae, the elution time is indicated, at which the detection is carried out, and the ordinates represent the relative intensity of the detected peaks.

A mixture of five dipeptides is introduced into the CPC column as detailed in example 18. FIG. 3 represents the recovery of the said five peptides at the output of the CPC column.

Five zones are shaded, each representing a different compound. From the left to the right of the elution profile, these zones are:

The first zone labeled GG represents the recovery of the dipeptide GG (GlycylGlycine) ranging from about 25 minutes to about 42 minutes after the purification was triggered.

The second zone labeled GY represents the recovery of the dipeptide GY (GlycylTyrosine) ranging from about 53 minutes to about 75 minutes after the purification was triggered.

The third zone labeled AY represents the recovery of the dipeptide AY (AlanylTyrosine) ranging from about 75 minutes to about 102 minutes after the purification was triggered.

The fourth zone labeled LV represents the recovery of the dipeptide LV (LeucylValine) ranging from about 142 minutes to about 152 minutes after the purification was triggered.

The fifth zone labeled LV represents the recovery of the dipeptide LV (Leucine-Valine) ranging from about 142 minutes to about 178 minutes after the purification was triggered.

FIG. 4 is a graphic representing the solubility of dipeptides in either an organic or an aqueous phase in function of the percentage of deprotonation of an exchanger dissolved in the aqueous phase. In abscissae, the percentage of deprotonation of the exchanger is represented; said abscissae are graduated from 0 to 30%. The ordinates represent a partition coefficient between the organic phase and the aqueous phase, said ordinates being graduated from 0 to 1.

Three curves are indicated on this graph, each representing a dipeptide.

The first curve is a full line with black lozenges. This curve represents the dipeptide GlycylGlycine.

The second curve is a full line with gray squares. This curve represents the dipeptide GlycylTyrosine.

The third curve is a full line with gray triangles. This curve represents the dipeptide LeucylValine.

FIG. 5 represents the elution profile of the compounds recorded by UV detection at 215 nm, at the output of a CPC column. In abscissae, the elution time at which the detection is carried out is represented, and in ordinate the relative intensity of the detected peaks is represented.

The elution profile indicates three distinct curves. Each curve represents the elution profile of an experiment.

A mixture of five dipeptides is introduced into the CPC column in the condition detailed in examples 27, 28 and 29. FIG. 5 represents the recovery of the said five peptides at the output of the CPC column for each of these experiments.

The curve on the top represents the elution profile obtained in example 42, with a concentration of HCl of 2.5 mM.

The curve in the middle represents the elution profile obtained in example 43, with a concentration of HCl of 3.5 mM.

The curve on the bottom represents the elution profile obtained in example 44, with a concentration of HCl of 5 mM.

FIG. 6 is an HPLC chromatogram of Alfalfa white protein hydrolysate. In abscissae, the retention time is indicated in minutes, and the ordinates represent the relative intensity of the peaks in a relative unit.

The HPLC chromatogram curve indicated in black line is recorded at a 215 nm wavelength, and the HPLC chromatogram curve indicated in gray line is recorded at a 280 nm wavelength.

The peak corresponding to the dipeptide VW (ValylTryptophane) is indicated on the chromatogram.

FIG. 7 is an HPLC chromatogram of Alfalfa white protein hydrolysate. In abscissae, the retention time is indicated in minutes, and the ordinates represent the relative intensity of the peaks in a relative unit. Chromatograms are recorded at a 215 nm wavelength.

The HPLC chromatogram curve indicated in black line represents the fraction which is purified by CPC, which is enriched in the dipeptide of interest (VW).

The HPLC chromatogram curve indicated in grey line represents the crude Alfalfa white protein hydrolysate prior to purification by CPC.

FIG. 8 is a HPLC chromatogram of peptide SF328 crude extract. In abscissae, the retention time is represented in minutes, and the ordinates represent the relative intensity of the peaks in a relative unit. The chromatogram is recorded at a 220 nm wavelength.

FIG. 9 is a HPLC chromatogram of peptide SF328 crude extract after purification by CPC in elution mode according to the example 54. In abscissae, the retention time is represented in minutes, and the ordinates represent the relative intensity of the peaks in a relative unit. The chromatogram is recorded at a 220 nm wavelength. (comparative example)

FIG. 10 is a HPLC chromatogram of peptide SF328 crude extract after purification by CPC in ion exchange mode according to the example 58. In abscissae, the retention time is represented in minutes, and the ordinates represent the relative intensity of the peaks in a relative unit. The chromatogram is recorded at a 220 nm wavelength.

FIG. 11 is a HPLC chromatogram of peptide SF328 crude extract after purification by CPC in ion exchange mode according to the example 59. In abscissae, the retention time is indicated in minutes, and the ordinates represent the relative intensity of the peaks in a relative unit. Chromatogram is recorded at 220 nm wavelength.

FIG. 12 is a proton NMR spectrum of the peptide SF328. In abscissae, the chemical displacement of the signal in ppm is represented.

FIG. 13 is a COSY NMR spectrum of the peptide SF328. COSY NMR spectrum indicates the correlations among the protons of a molecule in a two dimensional representation. On the upper and left side of the graphic the proton NMR spectrum of the peptide SF328 (see FIG. 12) is reproduced, and on the bottom and right side the chemical displacements are represented.

Each dot on the spectrum represents a correlation between two protons.

### EXPERIMENTAL

### Experimental Conditions

Solvents

All the solvents were purchased as chromatographic grade solvents from Carlo Erba (Rodano, Italy). Water was purified by de-ionization and reverse osmosis.

A biphasic system (1 L) was prepared by mixing the suitable solvents in the designated proportions in a separatory funnel. They were vigorously shaken and then allowed to settle until the phases became limpid.

Then an ionic exchanger was added in the organic phase in suitable concentrations. A displacer is also added to the aqueous mobile phase.

Chromatographic Apparatus

Separations were performed on a FCPC Kromaton Technologies apparatus (Angers, France) using a rotor made of 20 circular partition disks (1320 partition cells: 0.130 mL per cell; total column capacity: 205 mL, dead volume: 32.3 mL). Rotation speed could be adjusted from 200 to 2000 rpm, producing a centrifugal force field in the partition cell of ≈120 g at 1000 rpm and 480 g at 2000 rpm. The solvents were pumped by a Dionex P580HPG 4-way binary high-pressure gradient pump (Sunnyvale, Calif., USA).

The column was first filled with the organic stationary phase.

The samples were introduced into the column through a low-pressure injection valve (Upchurch, CIL Cluzeau, Sainte-Foy-La-Grande, France) equipped with a 10 mL sample loop. The aqueous mobile phase was then pumped in the descending mode. The flow rate was 2 mL/min and the rotation speed was 1200 rpm. The effluent detection was controlled by a Dionex UVD 170S detector at μ=220 nm equipped with a preparative flow cell (6 μL internal volume, path length of 2 mm). Fractions (2 mL) were collected by a Pharmacia Superfrac collector (Uppsala, Sweden). The experiments were conducted at room temperature (22±1° C.).

Analysis

The fraction quantification was performed on the customized Dionex Summit HPLC system, equipped with a P580 pump, an ASI-100 automated injector, a STH column oven and a UVD340S diode array detector and a Jupiter Proteo 90A (250 mm×4.6 mm id, 4 μm particle size) column with a security guard (Phenomenex, France). The elution was performed in the gradient mode with solvent A: 0.1% of trifluoroacetic acid in H₂O and solvent B: 0.09% of trifluoroacetic acid in CH₃CN (see below for the gradient profile). The flow rate was 1 mL/min. The wavelength UV detection was fixed at 220 nm. The temperature of the column oven was set at 40° C. The chromatographic data management was ensured by the Chromeleon software 6.0.1 version

Gradient:

| Time (min) | % A | % B |
|---|---|---|
| 0 | 85 | 15 |
| 30 | 55 | 45 |
| 35 | 55 | 45 |
| 36 | 85 | 15 |
| 40 | 85 | 15 |

Sample: 2.5 mg/mL in AcOH/Water (1:1, v/v)

Injection volume: 40 μl

¹H, ¹³C, COSY (correlation spectroscopy), HSQC (heteronuclear single quantum correlation) and HMBC (heteronuclear multiple bond correlation) NMR experiments were performed in CDCl₃. They were recorded on a Bruker (Wissembourg, France) Avance DRX 500 spectrometer (¹H at 500 MHz and ¹³C at 125 MHz).

### I Dipeptide Purification

Efficiency of the methodology for the CPC purification in ion exchange displacement mode of a peptide mixture was investigated on a mixture of five dipeptides: GlycylGlycine (GG), GlycylTyrosine (GY), AlanylTyrosine (AY), LeucylValine (LV), and LeucylTyrosine (LY).

20 mg to 200 mg of each peptide are introduced in the column.
These peptides have very close isoelectric points, ranging from 6.08 to 6.1 (according to EMBOSS), that implies they will be charged in the same way at the same pH. Nevertheless, those peptides covered a large “polarity” range from very polar GG to quite apolar LV and LY. GY and AY are peptides with a middle polar nature, which are moreover very close in a structural point a view. This mix allows the study of the selectivity of the process according to polarity but also to molecular structure.
GG (>99%) and GY (>99%) were purchased from Bachem (Bubendorf, Switzerland). AY (>99%), LV (>99%) and LY (>99%) were purchased from TCI Europe (Zwijndrecht, Belgium).

The purification of the peptide mixture was carried out
in a first time, with elution without displacement mode (this method is not part of the subject-matter of the present invention, and is presented here as a comparative example), this method proved to be inefficient (examples 1-6); and
in a second time, with elution in an ion exchange displacement mode, according to the invention, this method was successful (examples 7-38).

The ion exchange displacement mode was investigated to determine the best experimental conditions for CPC purification.

Parameters such as:
the cationic displacer concentration (examples 7-10),
the anionic exchanger concentration (examples 11-16),
the type of cationic displacer (examples 17-22),
the state of deprotonation of the anionic exchanger (example 23),
the segmentation of the CPC column (examples 24-26),
the concentration of H⁺ as a secondary cationic displacer (examples 27-30), and
the use of a cationic retainer and a anionic exchanger (examples 31-38), were investigated.

I1.1 Elution Mode

This method is not part of the present invention subject-matter and the following examples are presented here as a comparative examples.

### Example 1

The solvent system is MtBE/n-BuOH/Acetic acid/Water (1:4.5:1.5:6, v/v).

Rotation speed of the column is 1000 rpm.

Flow rate is 4 mL/min

Elution is in descending mode.

The peptide mass is 103.8 mg.

Stationary phase retention is 60%.

Product recovered: 98%

The peptide GlycylGlycine (GG) is separated from the peptide mixture.

The peptides GlycylTyrosine (GY) and AlanylTyrosine (AY) are not separated.

The peptides LeucylValine (LV) and LeucylTyrosine (LY) are not separated.

### Example 2

The solvent system is MtBE/n-BuOH/CH₃CN/Water 1% TFA (2:2:1:5, v/v).

Rotation speed of the column is 1000 rpm.

Flow rate is 3 mL/min

Elution is in descending mode.

The peptide mass is 100.2 mg.

Stationary phase retention is 65%.

Product recovered: 58%

The peptide GG is separated from the peptide mixture.

The peptides GY and AY are not separated.

The peptides LV and LY are not recovered from the column.

### Example 3

The solvent system is MtBE/n-BuOH/CH₃CN/Water 1% TFA (2:2:1:5, v/v).

Rotation speed of the column is 1000 rpm.

Flow rate is 3 mL/min

Elution is in dual mode (descending then ascending).

The peptide mass is 114.2 mg.

Stationary phase retention is 65%.

Product recovered: 88%

The peptide GG is separated from the peptide mixture.

The peptides GY and AY are not separated.

The peptides LV and LY are not fully recovered from the column.

### Example 4

The solvent system is MtBE/n-BuOH/CH₃CN/Water 1% TFA (3:1:1:5, v/v).

Rotation speed of the column is 1000 rpm.

Flow rate is 3 mL/min

Elution is in dual mode (descending then ascending).

The peptide mass is 108.3 mg.

Stationary phase retention is 65%.

Product recovered: 95%

The peptides GG and GY are not separated (GY in mixture with GG).

The peptides GY and AY are not separated.

The peptides LV and LY are not separated.

### Example 5

The solvent system is MtBE/n-BuOH/CH₃CN/Water 1% TFA (3:1:1:5, v/v).

Rotation speed of the column is 900 rpm.

Flow rate is 3 mL/min

Elution is in descending mode.

The peptide mass is 107.2 mg.

Stationary phase retention is 65%.

Product recovered: 99%

The peptides GG and GY are not separated (GY in mixture with GG).

The peptides GY and AY are not separated.

The peptides LV and LY are not separated.

### Example 6

The solvent system is MtBE/n-BuOH/CH₃CN/Water 1% TFA (2:2:1:5, v/v), then MtBE/n-BuOH/CH₃CN/Water 1% TFA (3:1:1:5, v/v)

Rotation speed of the column is 1000 rpm.

Flow rate is 3 mL/min.

Elution is in descending mode.

The peptide mass is 100.3 mg.

Stationary phase retention is 66%.

Product recovered: 97%

The peptides GG and GY are separated.

The peptides GY and AY are not separated.

The peptides LV and LY are not fully separated.

Elution mode, without ion exchange displacement, is not an option for the CPC purification of the studied peptide mixture, since no solvent system enabling a satisfactory separation between the five peptides was found.

I.2 Ion Exchange Mode

I.2.1 Displacer Concentration
The solvent system is MtBE/CH₃CN/n-BuOH/Water (2:1:2:5, v/v).
The ion-exchanger DEHPA concentration is 46.5 mM, and is partially deprotonated (5.15%) by triethylamine.
DEHPA/Peptide Ratio is 16.6.
The displacer is HCl.
Elution is in descending mode.
Rotation speed of the column is 1200 rpm.
Flow rate is 2 mL/min.

### Example 7

HCl concentration is 1 mM.

Stationary phase retention is 74%.

The peptides GG, GY and AY are separated. The separation is slow (200 minutes).

The peptides LV and LY are not recovered from the column.

### Example 8

HCl concentration is 5 mM.

Stationary phase retention is 75%.

The peptide GG is separated.

The peptides GY and AY are not fully separated.

The peptides LV and LY are not fully recovered from the column.

### Example 9

HCl concentration is 20 mM.

Stationary phase retention is 75%.

The peptide GG is separated.

The peptides GY and AY are not separated.

The peptides LV and LY are recovered from the column, but are not separated.

### Example 10

HCl concentration is 10 mM.

Stationary phase retention is 74%.

The peptide GG is separated.

The peptides GY and AY are not separated.

The peptides LV and LY are recovered from the column, but are not separated.

I.2.2 Exchanger Concentration

The solvent system is MtBE/CH₃CN/n-BuOH/Water (2:1:2:5, v/v).

The ion-exchanger is DEHPA.

The displacer is HCl (10 mM).

Elution is in descending mode.

Rotation speed of the column is 1200 rpm.

Flow rate is 2 mL/min.

### Example 11

DEPHA concentration is 30 mM.

DEHPA/peptide ratio is 10.7.

Stationary phase retention is 74%.

The peptide GG is separated.

The peptides GY and AY are not separated.

The peptides LV and LY are not separated.

### Example 12

DEPHA concentration is 20 mM.

DEHPA/peptide ratio is 7.1.

Stationary phase retention is 75%.

The peptide GG is separated.

The peptides GY and AY are not fully separated.

The peptides LV and LY are not fully separated.

### Example 13

DEPHA concentration is 10 mM.

DEHPA/peptide ratio is 3.6.

Stationary phase retention is 73%.

The peptide GG is separated.

The peptides GY and AY are not fully separated.

The peptides LV and LY are not fully separated.

### Example 14

DEPHA concentration is 5 mM.

DEHPA/peptide ratio is 1.8.

Stationary phase retention is 73%.

The peptides GG and GY are not fully separated.

The peptides GY and AY are not separated.

The peptide LV is not fully separated.

### Example 15

DEPHA concentration is 15 mM.

DEHPA/peptide ratio is 5.4.

Stationary phase retention is 73%.

Separation is good.

### Example 16

DEPHA concentration is 2 mM.

DEHPA/peptide ratio is 0.72.

Stationary phase retention is 73%.

No separation.

I.2.3 Displacer Type

The solvent system is MtBE/CH₃CN/n-BuOH/Water (2:1:2:5, v/v).

The ion-exchanger is DEHPA (15 mM).

DEHPA/peptide ratio is 5.4.

Elution is in descending mode.

Rotation speed of the column is 1200 rpm.

Flow rate is 2 mL/min.

### Example 17

The displacer is CaCl₂ (1.44 mM).

Secondary displacer is HCl (10 mM).

Experiment run time is 230 min

Stationary phase retention is 72%.

### Example 18

The displacer is CaCl₂ (1.44 mM).

Secondary displacer is HCl (10 mM).

Experiment run time is 180 min

Stationary phase retention is 72%.

FIG. 3 represents the chromatographic profile of example 18.

### Example 19

The displacer is CaCl₂ (5.4 mM).

Secondary displacer is HCl (10 mM).

Experiment run time is 230 min

Stationary phase retention is 73%.

### Example 20

The displacer is MgCl₂ (5 mM).

Secondary displacer is HCl (10 mM).

Experiment run time is 250 min

Stationary phase retention is 74%.

### Example 21

The displacer is MnCl₂ (5 mM, then 50 mM).

Secondary displacer is HCl (50 mM).

Experiment run time is 250 min

Stationary phase retention is 74%.

### Example 22

The displacer is KCl (10 mM, then 50 mM).

Secondary displacer is HCl (50 mM).

Experiment run time is 250 min.

Stationary phase retention is 75%.

For each experiment, peptides GY and AY are well separated.

For each experiment, peptides LV and LY require HCl in order to be eluted.

I.2.4 Deprotonated Exchanger

The Influence of the percentage of deprotonated exchanger over the CPC purification of dipeptides GG, GY and LV is determined.

DEHPA is deprotonated at 5.15%, 8%, 13%, 18% and 33%.

The Results are presented in FIG. 4.

An improvement of the peptide LV extraction is demonstrated when the exchanger is deprotonated to 30%.

### Example 23

The solvent system is MtBE/CH₃CN/n-BuOH/Water (2:1:2:5, v/v).
The ion-exchanger is DEHPA (15 mM, 33% deprotonated by triethylamine).
The displacer is CaCl₂ (1.44 mM).
CaCl₂ is added 10 minutes after the equilibrium state is reached. The equilibrium state is reached when no more stationary phase is forced out of the column (at the output of the column), by pumping the mobile phase in the column at the input of the column (34 minutes after the beginning of the purification).
Secondary displacer is HCl (10 mM).
HCl is added 30 minutes after the peptide GY is recovered (71 minutes after the beginning of the purification).
Elution is in descending mode.
Rotation speed of the column is 1200 rpm.
Flow rate is 2 mL/min
Stationary phase retention is 76%.
The peptides LV and LY are separated.

I.2.5 Segmented CPC Column
The solvent system is MtBE/CH₃CN/n-BuOH/Water (2:1:2:5, v/v).
The ion-exchanger is DEHPA (15 mM, 5.15% or 33% deprotonated by triethylamine).
The displacer is CaCl₂ (1.44 mM).
Secondary displacer is HCl (10 mM).
Elution is in descending mode.
Rotation speed of the column is 1200 rpm.
Flow rate is 2 mL/min.

### Example 24

The column is divided in two parts:
first half from the input to the middle of the column (100 mL) contains exchanger deprotonated at 5.15%,
second half from the middle of the column to the output (100 mL) contains exchanger deprotonated at 33%.

Stationary phase retention is 72%.
CaCl2 is added 10 minutes after the equilibrium state is reached (45 minutes after the beginning of the purification).
HCl is added 30 minutes after the peptide GY is recovered (105 minutes after the beginning of the purification).

The peptides GY and AY are separated.

The peptides LV and LY are not fully separated.

### Example 25

The column is divided in two parts:
first half from the input to the first quarter of the column (50 mL) contains exchanger deprotonated at 5.15%,
second half from the first quarter of the column to the output (150 mL) contains exchanger deprotonated at 33%.

Stationary phase retention is 76%.
CaCl₂ is added 10 minutes after the equilibrium state is reached (45 minutes after the beginning of the purification).
HCl is added 30 minutes after the peptide GY is recovered (105 minutes after the beginning of the purification).

The peptides GY and AY are separated.

The peptides LV and LY are not separated.

### Example 26

The column is divided in two parts:
first half from the input to the first quarter of the column (50 mL) contains exchanger deprotonated at 33%,
second half from the first quarter of the column to the output (150 mL) contains exchanger deprotonated at 5.15%.

Stationary phase retention is 76%.
CaCl₂ is added 10 minutes after the equilibrium state is reached (45 minutes after the beginning of the purification).
HCl is added 30 minutes after the peptide GY is recovered (105 minutes after the beginning of the purification).

The peptides GY and AY are separated.

The peptides LV and LY are almost separated.

I.2.6 HCl concentration

The solvent system is MtBE/CH₃CN/n-BuOH/Water (2:1:2:5, v/v).

The ion-exchanger is DEHPA (15 mM)

The displacer is CaCl₂ (1.44 mM).

Secondary displacer is HCl.

Elution is in descending mode.

Rotation speed of the column is 1200 rpm.

Flow rate is 2 mL/min.

### Example 27

DEHPA is 5.15% or 33% deprotonated by triethylamine).

The column is divided in two parts:
first half from the input to the first quarter of the column (50 mL) contains exchanger deprotonated at 33%,
second half from the first quarter of the column to the output (150 mL) contains exchanger deprotonated at 5.15%.

HCl concentration is 5 mM.

Stationary phase retention is 76%.

### Example 28

DEHPA is 5.15% or 33% deprotonated by triethylamine).

The column is divided in two parts:
first half from the input to the first quarter of the column (50 mL) contains exchanger deprotonated at 33%,
second half from the first quarter of the column to the output (150 mL) contains exchanger deprotonated at 5.15%.

HCl concentration is 2.5 mM.

Stationary phase retention is 77%.

The peptides LV and LY are separated.

### Example 29

DEHPA is 5.15% or 33% deprotonated by triethylamine).

Column is divided in two parts:
first half from the input to the first quarter of the column (50 mL) contains exchanger deprotonated at 33%,
second half from the first quarter of the column to the output (150 mL) contains exchanger deprotonated at 5.15%.

HCl concentration is 3.5 mM.

Stationary phase retention is 77%.

The peptides LV and LY are almost separated.

### Example 30

DEHPA is 5.15% deprotonated by triethylamine).

HCl concentration is 2.5 mM.

Stationary phase retention is 77%.

The peptides LV and LY are not separated.

FIG. 5 represents the chromatographic profiles of examples 27, 28 and 29.

I.2.7 Anionic Displacer and Cationic Exchanger

The solution pH is stabilized to 8 by adding a base in the solvent system. The peptides are in anionic form. The purification mixture contains the peptides GG, GY, AY, LV.

Elution is in descending mode.

Rotation speed of the column is 1200 rpm.

Flow rate is 2 mL/min.

### Example 31

The solvent system is AcOEt/n-BuOH/Water (3:2:5, v/v).
The ion-exchanger is aliquat (27.7 mM), aliquat is a quaternary ammonium salt, which alkyl chains are a mixture of C₈ (octyl) and C₁₀ (capryl) chains with C₈ predominating.

Aliquat/peptides ratio is 10.

The displacer is NaI (13.85 mM).

The base used is triethylamine.

Stationary phase retention is 72%.

The system is not stable, no purification is achieved.

### Example 32

The solvent system is AcOEt/n-BuOH/EtOH/Water (1:3:1:5, v/v).

The ion-exchanger is aliquat (27.7 mM).

Aliquat/peptides ratio is 10.

The displacer is NaI (13.85 mM).

The base used is triethylamine.

Stationary phase retention is 72%.

The system is not stable, no purification is achieved.

### Example 33

The solvent system is AcOEt/n-BuOH/Water (3:2:5, v/v).

The ion-exchanger is aliquat (27.7 mM).

Aliquat/peptides ratio is 10.

The displacer is NaI (13.85 mM).

The base used is NH₄OH.

Stationary phase retention is 73%.

The peptide LV is separated.

The peptides GG, GY and AY are not separated (not retained in the column).

### Example 34

The solvent system is AcOEt/n-BuOH/Water (3:2:5, v/v).

The ion-exchanger is aliquat (55.4 mM).

Aliquat/peptides ratio is 20.

The displacer is NaI (27.7 mM).

The base used is NH₄OH.

Stationary phase retention is 74%.

The peptide LV is separated.

The peptides GY and AY are not separated.

The peptide GG is not retained in the column.

### Example 35

The solvent system is AcOEt/n-BuOH/Water (3:2:5, v/v).

The ion-exchanger is aliquat (83.1 mM).

Aliquat/peptides ratio is 30.

The displacer is NaI (41.55 mM).

The base used is NH₄OH.

Stationary phase retention is 72%.

The peptides GG and GY are separated.

The peptides GY and AY are not separated.

The peptides AY and LV are not separated.

The peptide GG is not fully retained in the column.

### Example 36

The solvent system is AcOEt/n-BuOH/Water (2:3:5, v/v).

The ion-exchanger is aliquat (83.1 mM).

Aliquat/peptides ratio is 30.

The displacer is NaI (41.55 mM).

The base used is NH₄OH.

Stationary phase retention is 72%.

No purification is achieved.

### Example 37

The solvent system is AcOEt/n-BuOH/Water (4:1:5, v/v).

The ion-exchanger is aliquat (83.1 mM).

Aliquat/peptides ratio is 30.

The displacer is NaI (41.55 mM).

The base used is NH₄OH.

Stationary phase retention is 73%.

No purification is achieved.

### Example 38

The solvent system is AcOEt/Acetone/Water (3:2:5, v/v).

The ion-exchanger is aliquat (83.1 mM).

Aliquat/peptides ratio is 30.

The displacer is NaI (41.55 mM).

The base used is NH₄OH.

Stationary phase retention is 74%.

No purification is achieved.

Purification of dipeptides mixture by CPC in ion exchange mode with a anionic displacer and a cationic exchanger is not efficient.

### II Vegetal Extract Purification

Although the commercial value of Alfalfa as a forage plant is low, the Alfalfa products obtained through extraction/drying may be valorised in the cosmetic, nutraceutic or therapeutical domains.

Three different Alfalfa plant extracts were purified by ion exchange displacement mode CPC chromatography:
proteins from Alfalfa serum (examples 39-45),
an hydrolysate of Alfalfa xanthophyll proteins (examples 46-49), and
a dipeptide from Alfalfa white protein hydrolysate (examples 50-53).

II.1 Alfalfa Serum

The Alfalfa serum can be obtained according to the following process which comprises the following steps:

Alfalfa is ground and pressed to obtain oil cakes and a green juice.

The pH of the green juice is adjusted around 7.5-8 with NH₄OH, heated by adding water steam to a temperature around 85° C.

It is then centrifuged. The centrifugation enables to obtain two products: a concentrate of proteins and the Alfalfa serum.

The used Alfalfa serum is pre fractionated PEP-15 which is a concentrated Alfalfa serum. The alfalfa serum PEP-15 was kindly provided by Agro Industrie Recherche et Développement (ARD) (Route de Bazancourt, 51110 POMACLE, FRANCE)
Said serum has the following characteristics:

| | | |
|---|---|---|
| Dry Matter (DM) | (%) | >93% |
| Total nitrogened matter (N × 6.25) | (% DM) | 20% ± 2 |
| Mineral matter | (% DM) | 26% ± 2 |
| Total sugars (saccharose type) | (% DM) | 13% ± 1 |
| Total fibres | (% DM) | <5% |
| Free organic acids (lactic acid equivalent) | (% DM) | 8%± |
| C/N ratio | | 11 ± 1 |
| pH | (solution | 5.8 ± 0.2 |
| | at10%) | |

The peptidic distribution (%) is:

| | | |
|---|---|---|
| >10310 | daltons | 0.3 |
| 1140-10300 | daltons | 4.5 |
| 350-1140 | daltons | 44.5 |
| 130-350 | daltons | 34.6 |
| <130 | daltons | 16.1 |

The Aminogram is: Total (g/16 g N)

| | |
|---|---|
| Alanine | 3.58 |
| Arginine | 1.77 |
| Aspatic acid | 12.07 |
| Cystine | 0.95 |
| Glutamic acid | 5.82 |
| Glycine | 2.34 |
| Histidine | 0.95 |
| Isoleucine | 1.81 |
| Leucine | 2.82 |
| Lysine | 2.48 |
| Methionine | 0.38 |
| Phenylalanine | 2.00 |
| Proline | 2.96 |
| Sérine | 2.82 |
| Threonine | 2.53 |
| Tryptophane | 0.86 |
| Tyrosine | 1.53 |
| Valine | 2.39 |

The amino acid composition was expressed in g/16 g N because of the unknown conversion factors for each fraction from nitrogen to protein. If the customary conversion factor, or 6.25, is used, then g/16 g N is equivalent to g/100 g protein.
Before fractionation by the CPC process of the invention, the PEP-15 serum was pre fractionated. Polyphenols were removed by passing on an aromatic resin amberlite XAD-16 purchased from Sigma-Aldrich. Then saponins were removed by precipitation with acetone. The resulting supernatant was concentrated and freeze-dried and form the used alfalfa serum.

The best solvent system for purification of Alfalfa serum was investigated for different elution mode:
without displacement mode CPC (example 39) (this method is not part of the subject-matter of the present invention, and is presented here as a comparative example),
pH zone refining displacement mode CPC (example 40) (this method is not part of the subject-matter of the present invention, and is presented here as a comparative example), and
ion exchange displacement mode CPC according to the invention (example 41).

Purification of the Alfalfa serum was achieved by elution with ion exchange displacement mode CPC (examples 42-45).

The solubility of the Alfalfa serum was tested in different solvent system. The experiments were run in pillbox with small samples of solvents (total volume 2 mL) and small amounts of products (1 mg for example). The solubility of the Alfalfa serum peptides was checked by TLC (thin layer chromatography).

II.1.1 Elution Mode

This method is not part of the present invention subject-matter and the following example is presented here as a comparative example.

The classic solvent systems as Arizona (Pauli, G. F.; Pro, S. M.; Friesen, J. B., Countercurrent Separation of Natural Products. Journal of Natural Products 2008, 71 (8), 1489-1508.) or Acetone (Maciuk, A. Nouvelles méthodologies en chromatographie de partage liquide-liquide sans support solide: Application à l'isolement de substances naturelles. Thèse de doctorat, Université de Reims Champagne Ardenne, Reims, 2005). were investigated without success.

### Example 39

Polar solvent systems, typical for peptide purification by CPC, were investigated:

| | |
|---|---|
| n-BuOH/acetic acid/water | product remains in aqueous phase, |
| (4.3:1.4:4.3, v/v) | |
| AcOEt/CH₃CN/water (4:2:4, v/v) | product remains in aqueous phase, |
| n-BuOH/1-propanol/water | product remains in aqueous phase, |
| (4:2:4, v/v) | |
| n-BuOH/EtOH/water (5:2:6, v/v) | ⅕ of the product migrates to the |
| | organic layer, ⅘ remains in |
| | aqueous phase, |
| n-BuOH/water (1:1, v/v) | product remains in aqueous phase, |
| Isobutane/water (1:1, v/v) | product remains in aqueous phase. |

The elution mode is not an option for the CPC purification of Alfalfa serum peptides, since no satisfactory solvent system was found.

II.1.2 pH Zone Refining Mode

This method is not part of the present invention subject-matter and the following example is presented here as a comparative example.

The pH zone refining mode is a typical method for CPC purification of protected peptides.

### Example 40

The following solvent systems were investigated:

MtBE/n-BuOH/water (4:1:5, v/v)
acidic conditions: product remains in aqueous phase
basic conditions: product remains in aqueous phase

MtBE/n-BuOH/CH₃CN/water (2:2:1:5, v/v)
acidic conditions: product remains in aqueous phase
basic conditions: product remains in aqueous phase

MtBE/n-BuOH/CH₃CN/water (3:1:2:3, v/v)
acidic conditions: product remains in aqueous phase
basic conditions: product remains in aqueous phase

The pH zone refining mode is not an option for the CPC purification of Alfalfa serum peptides, since no satisfactory solvent system was found.

II.1.3 Ion Exchange Mode

This method is part of the invention.

### Example 41

The following solvent systems were investigated:

n-BuOH/acetic acid/water (4:1:4, 5, v/v)
exchanger: sodium bis(2-ethylhexyl) sulfosuccinate (AOT);
the peptides are extracted in the organic phase.

AcOEt/CH₃CN/water (6:11:11, v/v)
exchanger: sodium bis(2-ethylhexyl) sulfosuccinate (AOT);
the peptides are weakly extracted in the organic phase.

n-BuOH/EtOH/water (5:2:6, v/v)
exchanger: DEHPA;
the peptides are extracted in the organic phase.

AcOEt/n-BuOH/EtOH/water (1:3:1:5, v/v)
exchanger: DEHPA;
the peptides are extracted in the organic phase.

AcOEt/n-BuOH/EtOH/water (1:3:1:5, v/v)
exchanger: AOT;
the peptides are extracted in the organic phase when a large excess of AOT is used.

MtBE/n-BuOH/CH₃CN/water (2:2:1:5, v/v)
exchanger: DEHPA;
the peptides are extracted in the organic phase.

The ion exchange mode is adapted for the CPC purification of Alfalfa serum peptides, advantageous solvent system are AcOEt/n-BuOH/EtOH/water (1:3:1:5, v/v) and MtBE/n-BuOH/CH₃CN/water (2:2:1:5, v/v).

II.1.4 Ion Exchange Mode CPC Purification

This method is part of the invention.

The solvent system is MtBE/CH₃CN/n-BuOH/Water (2:1:2:5, v/v).
The lipophilic ion-exchanger is DEHPA (Bis (2-ethylhexyl)phosphate acid) and displacer is CaCl₂.

Elution is in descending mode.

Rotation speed of the column is 1200 rpm.

Flow rate is 2 mL/min.

### Example 42

The ion-exchanger DEHPA concentration is 31.3 mM, DEHPA is partially deprotonated (5.15%) by triethylamine.
The displacer CaCl₂ concentration is 3 mM.
The secondary displacer HCl (concentration 20.9 mM) is introduced after a first displacement with CaCl₂
The sample mass is 513.5 mg.
DEHPA/Peptide Ratio is 5.4.
Stationary phase retention is 74%.
Peptide isolation yield is 29%.

### Example 43

The ion-exchanger DEHPA concentration is 29.5 mM, DEHPA is partially deprotonated (2.15%) by triethylamine.
The displacer CaCl₂ concentration is 2.83 mM.
The sample mass is 561.8 mg.
DEHPA/Peptide Ratio is 5.4.
Stationary phase retention is 75%.
Peptide isolation yield is 29%.

### Example 44

The ion-exchanger DEHPA concentration is 88.5 mM, DEHPA is partially deprotonated (2.15%) by triethylamine.
The displacer CaCl₂ concentration is 8.48 mM.
The sample mass is 562.9 mg.
DEHPA/Peptide Ratio is 16.2.
Stationary phase retention is 75%.
Peptide isolation yield is 63%.

### Example 45

The ion-exchanger DEHPA concentration is 72 mM, DEHPA is partially deprotonated (33% then 5.15%) by triethylamine.
The displacer CaCl₂ concentration is 6.9 mM.
The sample mass is 503.7 mg.
DEHPA/Peptide Ratio is 32.
Stationary phase retention is 74%.
Peptide isolation yield is 91%.

II.2 Concentrated Hydrolysate of Xanthophyll Protein CPC Purification

Protein xanthophyll concentrate is a product of industrial alfalfa dehydration process used in cattle feed. This concentrate was hydrolysed by thermolysine.

Concentrated hydrolysate purification was carried out by
elution without displacement mode CPC (examples 46-47) (this method is not part of the subject-matter of the present invention, and is presented here as a comparative example), and
elution with ion exchange displacement mode CPC according to the invention, (examples 48-49).

II.2.1 Elution Mode

This method is not part of the present invention subject-matter and the following examples are presented here as a comparative examples.

Rotation speed of the column is 1000 rpm.

Flow rate is 3 mL/min.

### Example 46

The solvent system is MtBE/CH₃CN/n-BuOH/Water 1% TFA (2:1:2:5, v/v).

Elution is in ascending mode.

The sample mass is 500 mg.

Stationary phase retention is 70%.

No purification is achieved. Fractions are not well defined.

### Example 47

The solvent system is n-BuOH/Acetic acid/Water (4:1:5, v/v).
Elution is in dual mode, ascending then descending. The elution mode is inversed when the chromatogram comes back to the basal line (about after 2 hours of experimentation).
The sample mass is 508.9 mg.
Stationary phase retention is 75%.

No purification is achieved. Fractions are not well defined.

Elution mode, without ion exchange displacement, is not selective enough for CPC purification of the concentrated hydrolysate of xanthophyll proteins.

II.2.2 Ion Exchange Mode

This method is part of the invention.

The solvent system is MtBE/CH₃CN/n-BuOH/Water (2:1:2:5, v/v).

Rotation speed of the column is 1200 rpm.

Flow rate is 2 mL/min

Elution is in descending mode.

### Example 48

The ion-exchanger DEHPA concentration is 26.7 mM.
The exchanger is partially deprotonated (5.15%) by triethylamine.
The displacer CaCl₂ concentration is 2.56 mM.
The secondary displacer HCl (concentration 17.8 mM) is introduced after a first displacement with CaCl₂.
The sample mass is 509.9 mg.
Stationary phase retention is 74%.
Product recovered: 96%

### Example 49

The ion-exchanger DEHPA concentration is 36 mM.
The exchanger is partially deprotonated (33%, then 5.15%) by triethylamine.
The displacer CaCl₂ concentration is 3.45 mM.
The secondary displacer HCl (concentration 5.99 mM) is introduced after a first displacement with CaCl₂.
The sample mass is 251.8 mg.
Stationary phase retention is 75%.
Product recovered: 99%

II.3 Opioid Dipeptide CPC Purification in Ion Exchange Mode

Crude material is an Alfalfa white protein hydrolysate and ultrafiltrate from said hydrolysate.

Objective of the following examples (examples 50-53) is to purify or to selectively enrich one fraction with a dipeptide VW (ValylTryptophan) having an inhibitory capacity towards angiotensin converting enzyme (ACE) by elution with ion exchange displacement mode CPC. This method is part of the invention.

HLPC chromatogram of the crude extract is presented in FIG. 6.

The solvent system is MtBE/CH₃CN/n-BuOH/Water (2:1:2:5, v/v).

Rotation speed of the column is 1200 rpm.

Flow rate is 2 mL/min.

Elution is in descending mode.

The exchanger is partially deprotonated (30%, then 2.15%) by triethylamine.

### Example 50

The ion-exchanger DEHPA concentration is 94.4 mM.
The displacer CaCl₂ concentration is 9 mM.
The secondary displacer HCl (concentration 15.5 mM, then 30.7 mM) is introduced after a first displacement with CaCl₂.
Crude material is a protein hydrolysate.
The sample mass is 254.9 mg.
DEHPA/peptide ratio is 42.
Stationary phase retention is 75%.
Product recovered: 100%

### Example 51

The ion-exchanger DEHPA concentration is 11.2 mM.
The displacer CaCl₂ concentration is 1.06 mM.
The secondary displacer HCl (concentration 1.85 mM, then 2.78 mM) is introduced after a first displacement with CaCl₂.
Crude material is a protein hydrolysate.
The sample mass is 254.6 mg.
DEHPA/peptide ratio is 5.
Stationary phase retention is 76%.
Product recovered: 100%

### Example 52

The ion-exchanger DEHPA concentration is 33.6 mM.
The displacer CaCl₂ concentration is 3.18 mM.
The secondary displacer HCl (concentration 7.5 mM) is introduced after a first displacement with CaCl₂.
Crude material is a protein hydrolysate.
The sample mass is 252.9 mg.
DEHPA/peptide ratio is 15.
Stationary phase retention is 75%.
Product recovered: 100%

### Example 53

The ion-exchanger DEHPA concentration is 33.6 mM.
The displacer CaCl₂ concentration is 3.18 mM.
The secondary displacer HCl (concentration 7.5 mM) is introduced after a first displacement with CaCl₂.
Crude material is a protein hydrolysate ultrafiltrate.
The sample mass is 250.1 mg.
DEHPA/peptide ratio is 15.
Stationary phase retention is 74%.
Product recovered: 100%

HLPC chromatogram of the fraction containing the desired dipeptide VW obtained in example 53 is presented in FIG. 7.

### III Purification of a Peptide

The purification of the peptide H-Asp-Glu-Asn-Pro-Val-Val-His-Phe-Phe-Lys-Asn-Ile-Val-Thr-Pro-Arg-Thr-OH, named herein SF328 (dirucotide) was carried out by CPC.

SF 328 is an active drug in multiple sclerosis treatment (http://en.wikipedia.org/wiki/Dirucotide).

III.1. Peptide Synthesis
III.1.2 Peptide Assembly

SF328 is assembled by solid phase peptide synthesis starting from preloaded Fmoc-Thr(tBu)-Wang resin (3026 g; 0.7 mmol/g).

Suitably protected amino acids for Fmoc/tBu synthesis strategy have been used: Fmoc-Asp(tBu), Fmoc-Glu(OtBu), Fmoc-Asn(Trt), Fmoc-Pro, Fmoc-Val, Fmoc-His(Trt), Fmoc-Phe, Fmoc-Lys(Boc), Fmoc-Ile, Fmoc-Thr(tBu) and Fmoc-Arg(Pbf).

The excess of amino acids is 1.5 equivalents for all amino acids except for [Fmoc-Thr(tBu)]14 for which 2.5 equivalents excess has been used.

Coupling Steps:

The protected amino acids are coupled by using two types of reagents at room temperature.

Its completeness was determined by the ninhydrin (Kaiser) test.

The amino acids 1 to 13, 15 and 16 are coupled by successive addition of
HOBt (430 g) in DMF (7060 ml)
DIC (N,N′-Diisopropylcarbodiimide) (498 ml)
DIC (498 ml) Second addition after 1 hour
PyBop (benzotriazol-1-yl-oxytripyrrolidinophosphonium hexafluorophosphate) (1104.3 g), only in case of incomplete coupling after the second addition of DIC (positive Kaiser test).

Whereas Fmoc-Thr(tBu)]14 is coupled by adding successively the following reagents:
HOBt (143.3 g)
TBTU (O-(Benzotriazol-1-yl)-N,N,N′,N′-tetramethyluronium tetrafluoroborate) (1703.3 g) in DMF (Dimethylformamide) (3760 ml)
TBTU (1703.3 g) in DMF (3760 ml) Second addition after 1 hour
TBTU (681.3 g) in DMF (1880 ml) only in case of incomplete coupling after the second addition of TBTU (positive Kaiser test).

After each coupling step, the peptide-resin is washed 8 times with 18156 ml of DMF each.

Fmoc Deprotection Steps:

The Fmoc deprotection steps are performed by using two types of piperidine based mixtures. at room temperature.

For amino acids 4 to 17, Fmoc protecting group is cleaved by using 18156 ml of a mixture composed of 5% piperidine, 1% DBU, 89% DMF and 5% BTMACl (benzyltrimethylammonium chloride) in methanol. The completeness of the reaction is monitored by NIR (Near Infra Red Spectroscopy) by measurement of stability of the level of DBF (dibenzofulvene) in the reaction mixture.

For amino acids 1 to 3, Fmoc protecting group is cleaved by using 18156 ml of a mixture consisting in 20% piperidine and 80% of 0.1M HOBt in DMF.

After each Fmoc cleavage, the peptide resin is washed 8 times with 18156 ml of DMF each.

When the peptide is completely assembled, the peptide resin is washed with DMF (8 times 18156 ml) and DCM (dichloromethane) (6 times 18156 ml). It is then dried under vacuum (around 20 mbars) at 25° C. until constant weight (9055 g). Yield: 96.8%.

III.1.2 Global Deprotection

While stirring, the dried peptide resin (9055 g) is progressively added to a cooled mixture (10° C.) composed of TIS (triisopropylsilane) (2.7 L), water (2.7 L) and TFA (103.2 L).

At the end of resin addition, the temperature is set at 20° C. and the suspension is stirred during 3 hours.

The suspension is filtered and the resin is washed 3 times with 53.4 L of the cleavage mixture.

The combined filtrates are evaporated under reduced pressure until a residual volume of about 27 L.

This concentrated solution is poured into cooled (7° C.) diisopropyl ether (89 L) in order to precipitate the crude SF328.

The precipitate is filtered as soon as possible and washed 7 times with diisopropylether (about 13 L each).

The crude SF328 is dried under vacuum (Around 20 mbars) at a temperature not exceeding 30° C. until constant weight (4.171 Kg)

Yield: 68%; purity: 86%

III.1. Peptide Purification

Considering the peptidic nature of SF328, both the elution mode and displacement mode were investigated. The ion-exchange mode was particularly developed, as recent works have shown its efficiency for peptide purification.

Solvent partition of peptide SF 328 was evaluated and purification was carried out by
elution without displacement mode CPC (example 54) (this method is not part of the subject-matter of the present invention, and is presented here as a comparative example), and
elution with ion exchange displacement mode CPC according to the invention, (examples 55-60).
   HPLC of the SF328 crude sample (prior to CPC purification)

| | Ret. Time | Height | Area | Rel. Area |
|---|---|---|---|---|
| No. | min | mAU | mAU*min | % |
| 1 | 6.22 | 6.190 | 1.733 | 0.23 |
| 2 | 7.44 | 3.740 | 1.169 | 0.16 |
| 3 | 8.17 | 7.021 | 1.115 | 0.15 |
| 4 | 9.15 | 5.168 | 1.970 | 0.27 |
| 5 | 9.82 | 2.897 | 1.265 | 0.17 |
| 6 | 10.69 | 3.625 | 1.157 | 0.16 |
| 7 | 11.85 | 24.743 | 4.392 | 0.59 |
| 8 | 12.24 | 3.411 | 0.456 | 0.06 |
| 9 | 13.45 | 2.055 | 1.211 | 0.16 |
| 10 | 13.92 | 10.529 | 1.212 | 0.16 |
| 11 | 14.41 | 1.654 | 0.411 | 0.06 |
| 12 | 15.39 | 2.715 | 0.287 | 0.04 |
| 13 | 16.44 | 3.763 | 1.477 | 0.20 |
| 14 | 18.59 | 11.926 | 16.488 | 2.23 |
| 15 | 20.03 | 50.395 | 18.179 | 2.46 |
| 16 | 20.31 | 76.029 | 24.494 | 3.31 |
| 17 | 21.00 | 1704.101 | 631.812 | 85.33 |
| 18 | 22.10 | 51.556 | 24.684 | 3.33 |
| 19 | 24.41 | 10.677 | 2.134 | 0.29 |
| 20 | 26.05 | 3.331 | 0.768 | 0.10 |
| 21 | 26.86 | 2.527 | 0.501 | 0.07 |
| 22 | 27.88 | 14.733 | 2.537 | 0.34 |
| 23 | 29.82 | 4.569 | 0.958 | 0.13 |
| Total: | | 2007.354 | 740.410 | 100.00 |

The related HPLC spectrum is presented in FIG. 8.

III.3 Elution without Displacement Mode

This method is not part of the present invention subject-matter and the following example is presented here as a comparative example.

Selection of the Biphasic Solvent System

Partition coefficient of SF328 was evaluated by TLC in different biphasic solvent systems. In the elution mode, it is required that the KD value of the compound of interest lies between 0.2 and 5 (preferentially about 1).

| | KD for SF328 | | |
|---|---|---|---|
| | Upper | Lower | Evaluation |
| Biphasic solvent system | phase | phase | KD |
| BuOH/Acetic acid/Water (4:1:5, v/v) | + | +++ | average |
| BuOH/Acetic acid/Water (5:1:4, v/v) | + | ++++ | poor |
| MtBE/CH₃CN/BuOH/Water 1% TFA | ++ | ++ | good |
| (2:1:2:5, v/v) | | | |
| MtBE/CH₃CN/Water (2:2:3, v/v) | − | +++ | poor |
| MtBE/CH₃CN/Water (3:2:5, v/v) | ++ | +++ | average |

### CPC Run

### Example 54

Only the system MtBE/CH₃CN/BuOH/Water 1% TFA (2:1:2:5, v/v) was tested. The experimental conditions were:
Rotation speed: 1300 rpm.
Sample injected: 100 mg.
Elution mode: ascending.
Flow rate: 8 ml/min.

This system causes the degradation of SF328.

HPLC of the SF328 Sample Purified by Elution

| | Ret. Time | Height | Area | Rel. Area |
|---|---|---|---|---|
| No. | min | mAU | mAU*min | % |
| 1 | 5.97 | 3.210 | 2.480 | 0.31 |
| 2 | 8.56 | 2.296 | 1.524 | 0.19 |
| 3 | 8.95 | 5.440 | 0.468 | 0.06 |
| 4 | 9.59 | 2.498 | 0.890 | 0.11 |
| 5 | 11.10 | 2.560 | 2.643 | 0.33 |
| 6 | 15.60 | 2.958 | 6.451 | 0.81 |
| 7 | 21.21 | 746.514 | 451.896 | 56.80 |
| 8 | 23.78 | 423.808 | 329.246 | 41.38 |
| Total: | | 1189.284 | 795.600 | 100.00 |

The related HPLC spectrum is presented in FIG. 9.

III.4 Elution with Ion Exchange Displacement Mode

This method is part of the invention.

Purification Parameters
The solvent system is MtBE/CH₃CN/BuOH/Water (2:1:2:5, v/v).
The lipophilic ion-exchanger is DEHPA (Bis (2-ethylhexyl)phosphate acid) and displacer is CaCl₂.
The exchanger is partially deprotonated (5.15%) by triethylamine.
Elution is in descending mode.
Rotation speed of the column is 1200 rpm.
Flow rate is 2 mL/min.

### CPC Runs

### Example 55

The ion-exchanger DEHPA concentration is 1.5 mM.

The displacer CaCl₂ concentration is 0.14 mM.

The sample mass is 104.4 mg.

Addition displacer NO.

Product recovered: 19 mg (yield 75%, recovery 22%, purity 89%).

The yield is the total balance in mass

The recovery is the ratio SF328 pur de la fraction/SF328 pur injecté

Solvent consumption: 200 mL for the stationary phase,
140 mL for the mobile phase.

Productivity 12.6 mg/hour.

### Example 56

The ion-exchanger DEHPA concentration is 15 mM.

The displacer CaCl₂ concentration is 1.4 mM.

The sample mass is 101.4 mg.

Addition displacer NO.

Product recovered: 30 mg (yield 50%, recovery 35%, purity 91%).

Solvent consumption: 200 mL for the stationary phase,
140 mL for the mobile phase.

Productivity 20 mg/hour.

### Example 57

The ion-exchanger DEHPA concentration is 30 mM.
The displacer CaCl₂ concentration is 2.8 mM.
The sample mass is 99.8 mg.
Addition of the displacer 18 minutes after the equilibrium state is reached. The equilibrium state is reached when no more stationary phase is forced out of the column (at the output of the column), by pumping the mobile phase in the column at the input of the column
Product recovered: 30 mg (yield 85.5%, recovery 36%, purity 94%).
Solvent consumption: 200 mL for the stationary phase,
   180 mL for the mobile phase.
Productivity 15 mg/hour.

### Example 58

The ion-exchanger DEHPA concentration is 30 mM.

The displacer CaCl₂ concentration is 2.8 mM.

The sample mass is 98.2 mg.

Addition of the displacer 5 minutes after the equilibrium state is reached.

Product recovered: 27 mg (yield 100%, recovery 33%, purity 97%).

Solvent consumption: 200 mL for the stationary phase,
180 mL for the mobile phase.

Productivity 13.5 mg/hour.

HPLC of the Purified SF328 Peptide

| | Ret. Time | Height | Area | Rel. Area |
|---|---|---|---|---|
| No. | min | mAU | mAU*min | % |
| 1 | 7.55 | 4.605 | 0.536 | 0.06 |
| 2 | 10.89 | 1.013 | 0.098 | 0.01 |
| 3 | 13.94 | 5.579 | 0.708 | 0.08 |
| 4 | 16.35 | 1.070 | 0.299 | 0.03 |
| 5 | 20.58 | 89.052 | 21.635 | 2.47 |
| 6 | 21.03 | 2086.473 | 853.423 | 97.34 |
| Total: | | 2187.791 | 876.700 | 100.00 |

The related HPLC spectrum is presented in FIG. 10.

### Example 59

The ion-exchanger DEHPA concentration is 60 mM.

The displacer CaCl₂ concentration is 5.6 mM.

The sample mass is 197.5 mg.

Addition of the displacer 5 minutes after the equilibrium state is reached

Product recovered: 80 mg (yield 96%, recovery 48%, purity 98%).

Solvent consumption: 200 mL for the stationary phase,
160 mL for the mobile phase.

Productivity 44.4 mg/hour.
HPLC of the Purified SF328 Peptide

| | Ret. Time | Height | Area | Rel. Area |
|---|---|---|---|---|
| No. | min | mAU | mAU*min | % |
| 1 | 8.61 | 3.071 | 1.501 | 0.34 |
| 2 | 9.69 | 3.286 | 1.474 | 0.34 |
| 3 | 11.47 | 1.824 | 0.498 | 0.11 |
| 4 | 13.71 | 7.921 | 1.014 | 0.23 |
| 5 | 17.36 | 1.956 | 0.725 | 0.16 |
| 6 | 18.68 | 1.086 | 0.232 | 0.05 |
| 7 | 20.94 | 1028.942 | 434.085 | 98.76 |
| Total: | | 1048.085 | 439.528 | 100.00 |

The related HPLC spectrum is presented in FIG. 11.

The peptide SF328 obtained in this experiment is analysed by NMR spectroscopy (see FIGS. 12 and 13).

### Example 60

The ion-exchanger DEHPA concentration is 120 mM.

The displacer CaCl₂ concentration is 11.1 mM.

The sample mass is 405.5 mg.

Addition of the displacer 5 minutes after the equilibrium state is reached.

Product recovered: 204.1 mg (yield 94%, recovery 60%, purity 98%).

Solvent consumption: 200 mL for the stationary phase,
160 mL for the mobile phase.

Productivity 113.4 mg/hour.

### Example 61

The ion-exchanger DEHPA concentration is 300 mM.

The displacer CaCl₂ concentration is 27.8 mM.

The sample mass is 1.0043 g.

Addition of the displacer 5 minutes after the equilibrium state is reached.

Product recovered: 586.9 mg (yield 100%, recovery 69%, purity 97%).

Solvent consumption: 200 mL for the stationary phase,
160 mL for the mobile phase.

Productivity 320.2 mg/hour.

1-28. (canceled)

29. A process for purifying at least one product from a substrate containing said at least one product, comprising subjecting said substrate to centrifugal partition chromatography in an ion exchange displacement mode to purify said at least one product from said substrate, said at least one product being an amphoteric product.

30. The process according to claim 29, wherein said substrate is contained in a chromatographic mixture, said chromatographic mixture containing also a solvent mixture, at least one displacer, at least one exchanger, and at least one retainer.

31. The process according to claim 29, wherein said at least one product to be purified is at least one protein or one peptide or peptide derivative, in particular a protected peptide, or is at least one amino acid, natural or not, protected or not.

32. The process according to claim 30, wherein said at least one displacer and said at least one retainer are cationic, and said at least one exchanger is anionic.

33. The process according to claim 30, wherein several displacers and one exchanger are used.

34. The process according to claim 30, wherein one displacer and one exchanger at various percentages of deprotonation are used, and said percentages of deprotonation varying in particular from 1% to 50%.

35. The process according to claim 30, wherein, wherein several displacer and one exchanger at various percentages of deprotonation are used.

36. A process for the purification of at least one product to be purified from a substrate by centrifugal partition chromatography in an ion exchange displacement mode, said product to be purified being an amphoteric product, wherein said process comprises:
at least one step of rotation of a centrifugal partition chromatography column, said column comprising a chromatographic mixture containing said substrate, a biphasic solvent mixture, at least one displacer, at least one exchanger and at least one retainer,
said biphasic solvent mixture being constituted by two non-miscible phases, one phase being the stationary phase and the other phase being the mobile phase, and
at least one step of pumping of said aqueous mobile phase through said column,
for a time sufficient to purify said product to be purified,
a step of recovery of said at least one product to be purified in a purified form.

37. A process for the purification of at least one product to be purified from a substrate by centrifugal partition chromatography in an ion exchange displacement mode, said product to be purified being an amphoteric product, wherein said process comprises:
at least one step of rotation of a centrifugal partition chromatography column, said column comprising a chromatographic mixture containing said substrate, a biphasic solvent mixture, several displacers, one exchanger and at least one retainer, said biphasic solvent mixture being constituted by two non-miscible phases, one phase being the stationary phase and the other phase being the mobile phase, and
at least one step of pumping of said aqueous mobile phase through said column,
for a time sufficient to purify said product to be purified,
a step of recovery of said at least one product to be purified in a purified form.

38. A process for the purification of at least one product to be purified from a substrate by centrifugal partition chromatography in an ion exchange displacement mode, said product to be purified being an amphoteric product, wherein said process comprises:
at least one step of rotation of a centrifugal partition chromatography column, said column comprising a chromatographic mixture containing said substrate, a biphasic solvent mixture, one displacer and one exchanger at various percentages of deprotonation are used, said percentages of deprotonation varying in particular from 1% to 50% and at least one retainer, said biphasic solvent mixture being constituted by two non-miscible phases, one phase being the stationary phase and the other phase being the mobile phase, and
at least one step of pumping of said aqueous mobile phase through said column,
for a time sufficient to purify said product to be purified,
a step of recovery of said at least one product to be purified in a purified form.

39. A process for the purification of at least one product to be purified from a substrate by centrifugal partition chromatography in an ion exchange displacement mode, said product to be purified being an amphoteric product, wherein said process comprises:
at least one step of rotation of a centrifugal partition chromatography column, said column comprising a chromatographic mixture containing said substrate, a biphasic solvent mixture, several displacers and one exchanger, wherein one displacer and one exchanger at various percentages of deprotonation are used, said percentages of deprotonation varying in particular from 1% to 50%,
said biphasic solvent mixture being constituted by two non-miscible phases, one phase being the stationary phase and the other phase being the mobile phase, and
at least one step of pumping of said aqueous mobile phase through said column,
for a time sufficient to purify said product to be purified,
a step of recovery of said at least one product to be purified in a purified form.

40. The process according to claim 36, wherein said at least one product to be purified is a protein, and said protein contains from 1000 to 100 amino acids, preferably from 1000 to 300 amino acids, preferably from 500 to 100 amino acids, preferably from 1000 to 500 amino acids, preferably from 500 to 300 amino acids, preferably from 300 to 100 amino acids, or wherein said at least one product to be purified is a peptide or is a peptide derivative, in particular a protected peptide, or is an amino acid, natural or not, protected or not.

41. The process according to claim 36, wherein said at least one product to be purified contains less than about 80% polar amino acids, preferably less than about 70% polar amino acids, preferably less than about 60% polar amino acids, preferably less than 50% polar amino acids.

42. The process according to claim 36, constituted by 2 to 5 different solvents, preferably 3 different solvents, particularly 4 different solvents, and in particular wherein water and n-butanol are two of the solvents constituting said biphasic solvent mixture; and in particular
wherein one of the solvents contained in said biphasic solvent mixture is a solvent less polar than n-butanol, such as alkanes, ethyl acetate, chlorinated solvent, lipophilic esters, lipophilic ketones, lipophilic ethers; or
wherein one of the solvents contained in said biphasic solvent mixture is a solvent miscible with both H₂O and n-butanol, such as methanol, ethanol, propanol, acetonitrile, acetone.

43. The process according to claim 36, wherein said biphasic solvent mixture contains the following four solvents:
water, and
a solvent miscible with both H₂O and n-butanol, such as methanol, ethanol, acetonitrile, acetone and
n-butanol, and
a solvent less polar than n-butanol, such as alkanes, ethyl acetate, chlorinated solvent, lipophilic esters, lipophilic ketones, lipophilic ethers.

44. The process according to claim 36, wherein said at least one exchanger is anionic, and said retainer is cationic.

45. The process according to claim 36, wherein said at least one exchanger is an alkylated phosphoric acid derivative, particularly DEHPA.

46. The process according to claim 36, wherein said at least one displacer is cationic.

47. The process according to claim 36, comprising:
a step of introduction of the organic stationary phase in the Centrifugal Partition Chromatography column, said organic stationary phase comprising at least one exchanger and at least one retainer, and
a step of introduction of the substrate containing at least one product to be purified in the said Centrifugal Partition Chromatography column, and
a step of introduction of the aqueous mobile phase in the Centrifugal Partition Chromatography column, said mobile phase comprises at least one displacer, and
a step of pumping the said aqueous mobile phase through the said Centrifugal Partition Chromatography column, in order to enable the mobilization of the said at least one product to be purified through the said column, and
a step of recovery of at least one of the said products to be purified in a purified form,
said column being in rotation from the introduction of the substrate to the recovery of at least one of the said products to be purified in a purified form.

48. The process according to claim 36, comprising:
a step of introduction of the organic stationary phase in the Centrifugal Partition Chromatography column, said organic stationary phase comprising one exchanger, and at least one retainer, and
a step of introduction of the substrate containing at least one product to be purified in the said Centrifugal Partition Chromatography column, and
a step of introduction of the aqueous mobile phase in the Centrifugal Partition Chromatography column, said mobile phase comprises several displacers, and
a step of pumping the said aqueous mobile phase through the said Centrifugal Partition Chromatography column, in order to enable the mobilization of the said at least one product to be purified through the said column, and
a step of recovery of at least one of the said products to be purified in a purified form,
said column being in rotation from the introduction of the substrate to the recovery of at least one of the said products to be purified in a purified form.

49. The process according to claim 36, comprising:
a step of introduction of the organic stationary phase in the Centrifugal Partition Chromatography column, said organic stationary phase comprising one displacer and one exchanger at various percentages of deprotonation, said percentages of deprotonation varying in particular from 1% to 50% and at least one retainer, and
a step of introduction of the substrate containing at least one product to be purified in the said Centrifugal Partition Chromatography column and
a step of introduction of the aqueous mobile phase in the Centrifugal Partition Chromatography column, said mobile phase comprises at least one displacer, and
a step of pumping the said aqueous mobile phase through the said Centrifugal Partition Chromatography column, in order to enable the mobilization of the said at least one product to be purified through the said column, and
a step of recovery of at least one of the said products to be purified in a purified form,
said column being in rotation from the introduction of the substrate to the recovery of at least one of the said products to be purified in a purified form.

50. The process according to claim 36, comprising:
a step of introduction of the organic stationary phase in the Centrifugal Partition Chromatography column, said organic stationary phase comprising one displacer and one exchanger at various percentages of deprotonation, said percentages of deprotonation varying in particular from 1% to 50%, and at least one retainer, and
a step of introduction of the substrate containing at least one product to be purified in the said Centrifugal Partition Chromatography column and
a step of introduction of the aqueous mobile phase in the Centrifugal Partition Chromatography column, said mobile phase comprises several displacers, and
a step of pumping the said aqueous mobile phase through the said Centrifugal Partition Chromatography column, in order to enable the mobilization of the said at least one product to be purified through the said column, and
a step of recovery of at least one of the said products to be purified in a purified form,
said column being in rotation from the introduction of the substrate to the recovery of at least one of the said products to be purified in a purified form.

51. The process according to claim 36, comprising:
at least one step of triggering the rotation of a Centrifugal Partition Chromatography column, said column comprising a separation mixture comprising said biphasic solvent mixture, said at least one retainer, said at least one exchanger, possibly said substrate, and possibly said at least one displacer,
wherein said centrifugal partition chromatography column comprises:
at one end, an input where both aqueous and organic phases, said retainer, said exchanger, possibly said substrate and possibly said displacer, are introduced in the column at an appropriate time, and
at the other end, an output where said organic stationary phase, said aqueous mobile phase, said retainer, said exchanger, possibly said products to be purified in a purified form, and possibly said displacer, are recovered from the column, and
wherein said displacement Centrifugal Partition Chromatography process allows the formation two to 2n+3 zones within the centrifugal partition chromatography column, and wherein n is the number of the different products to be purified from the substrate: —possibly a head zone, contiguous to the output of the column, said head zone comprising said retainer and said exchanger dissolved in said stationary phase,
possibly a tail zone, contiguous to the input of the column, wherein said tail zone comprises “displacer—exchanger” ion pairs,
central zones, situated between the head zone and the tail zone, or between the input and the output of the said column if respectively no tail zone or head zone are present,
wherein the number of said central zones ranges from 2n+1, wherein
the first central zone is the zone, among the central zones, which is the closest to the output of the column, or which is contiguous to the head zone if said head zone is present, and
the last central zone, is the zone, among the central zones, which is the closest to the input of the column, or which is contiguous to the tail zone if said tail zone is present, and
wherein, independently from each other, each of said central zones comprises or not, at least one “exchanger—product to be purified” ion pair,
providing that at least one central zone comprises at least one product to be purified,
preferably, at least one of the said product to be purified is located in a central zone containing no other product to be purified,
preferably each of said n products to be purified is located in a central zone containing no other product to be purified.

52. The process according to claim 36,
wherein said displacement Centrifugal Partition Chromatography process allows the formation of two to 2n+2 zones within the centrifugal partition chromatography column, and wherein n is the number of the different products to be purified from the substrate, said zones consisting in: —a head zone, contiguous to the output of the column, said head zone comprising said retainer and said exchanger dissolved in said stationary phase, and
central zones, situated between the head zone and the output of the said column, wherein
the number of said central zones ranges from 1 to 2n+1,
the first central zone is the zone, among the central zones, which is the closest to the output of the column, and
the last central zone, is the zone, among the central zones, which is the closest to the input of the column,

53. The process according to claim 36,
Wherein said displacement Centrifugal Partition Chromatography process allows the formation of n+2 to 2n+3 zones within the centrifugal partition chromatography column, and wherein n is the number of the different products to be purified from the substrate, said zones consisting in: —a head zone, contiguous to the output of the column, said head zone comprising said retainer and said exchanger dissolved in said stationary phase, and
a tail zone, contiguous to the input of the column, wherein said tail zone comprises “displacer—exchanger” ion pairs, and
central zones, situated between the head zone and the tail zone,
wherein
the number of said central zones ranges from 1 to 2n+1,
the first central zone is the zone, among the central zones, which is the closest to the output of the column, and
the last central zone, is the zone, among the central zones, which is the closest to the input of the column.

54. The process according to claim 36, comprising:
a step of introduction of the organic stationary phase in the Centrifugal Partition Chromatography column, said organic stationary phase comprising at least one exchanger and at least one retainer, and said organic phase comprising the following four solvents:
water less than 15% of the volume of the organic phase
a solvent miscible with both H₂O and n-butanol, such as methanol, ethanol, acetonitrile, acetone at a volumic proportion less than 50%, and
n-butanol (5 to 90%, v/v), and
a solvent less polar than n-butanol (5 to 90% v/v), such as alkanes, ethyl acetate, chlorinated solvent, lipophilic esters, lipophilic ketones, lipophilic ethers, and
a step of introduction of the substrate comprising at least one product to be purified in the said Centrifugal Partition Chromatography column, and
a step of continuous introduction of the aqueous mobile phase in the Centrifugal Partition Chromatography column, said aqueous mobile phase comprising the following three solvents:
water, and
a solvent miscible with both H₂O and n-butanol, such as methanol, ethanol, acetonitrile, acetone or a mixture of them and
possibly n-butanol, and
a solvent less polar than n-butanol, such as alkanes, ethyl acetate, chlorinated solvent, lipophilic esters, lipophilic ketones, lipophilic ethers,
a step of continuous introduction of a least one displacer in said aqueous mobile phase, and pumping the aqueous mobile phase comprising the at least one displacer through the said Centrifugal Partition Chromatography column, in order to enable the mobilization of the said at least one product to be purified through the said column, and
a step of continuous recovery of at least one of the said products to be purified in a purified form, at the output of the column,
wherein the continuous rotation of the column, the continuous introduction of a least one displacer in said aqueous phase, and the continuous pumping of said aqueous phase through the column, are maintained,
said step of continuous recovery being triggered when the first central batch is recovered at the output of the column.

55. The process according to claim 36, comprising:
a step of introduction of the organic stationary phase in the Centrifugal Partition Chromatography column, said organic stationary phase comprising at least one exchanger and at least one retainer, and said organic phase comprising: acetonitrile, n-butanol, MtBE, and traces of water, and
a step of introduction of the substrate comprising a peptide or a peptide derivative to be purified in particular the SF 328 to be purified in the said Centrifugal Partition Chromatography column, and
a step of continuous introduction of the aqueous mobile phase in the Centrifugal Partition Chromatography column, and said aqueous mobile phase comprising water, acetonitrile, n-butanol, and traces of MtBE,
a step of continuous introduction of a least one displacer in said aqueous mobile phase, and pumping the aqueous mobile phase comprising the at least one displacer through the said Centrifugal Partition Chromatography column, in order to enable the mobilization of the said at least one product to be purified through the said column, and
a step of continuous recovery of the peptide or peptide derivative to be purified in particular the SF 328 to be purified in a purified form, at the output of the column,
wherein the continuous rotation of the column, the continuous introduction of a least one displacer in said aqueous phase, and the continuous pumping of said aqueous phase through the column, are maintained,
said step of continuous recovery being triggered when the first central batch is recovered at the output of the column.

56. The process according to claim 36, wherein
the step of continuous introduction of the aqueous mobile phase in the Centrifugal Partition Chromatography column, and the step of continuous introduction of a least one displacer in said aqueous phase, are repeated from 2 to 4 cycles,
said cycles being in a number sufficient to recover all the products to be purified,
wherein, for a given cycle, the displacer used is different from the displacer used in the previous cycles, and enables the formation of a “retainer—exchanger” ion pair with an affinity higher than the affinity of at least one of the “exchanger—product to be purified” ion pairs that remain in the column,
in order to enable, for each cycle, the recovery at the output of the column of at least one batch comprising at least one of the said product to be purified, remaining in the column, in a purified from.
